(19) United States
(12) Patent Application Publication
(10) Pub. No.: US 2022/0047698 A1
(43) Pub. Date: Feb. 17, 2022

(54) SYNTHETIC INNATE IMMUNE RECEPTOR LIGANDS AND USES THEREOF

(71) Applicant: ALBERTA RESEARCH CHEMICALS INC., Edmonton (CA)

(72) Inventors: Damayanthi YALAMATI, Edmonton, AB (CA); Praveen BARADI, Edmonton, AB (CA); R. Rao KOGANTY, Edmonton, AB (CA)

(21) Appl. No.: 17/275,603

(22) PCT Filed: Apr. 29, 2019
(86) PCT No.: PCT/CA2019/050555

§ 371 Date: Mar. 11, 2021

Related U.S. Application Data
(60) Provisional application No. 62/751,166, filed on Oct. 26, 2018.

(51) Int. Cl. A61K 39/39; A61K 9/16

(52) U.S. Cl. CPC A61K 39/39; A61K 2039/55516; A61K 9/1647

(57) ABSTRACT

An adjuvant formulation includes a monophosphoryl Lipid A (MPLA) analogue, a Pam3CSK4 analogue, or a muramyldipeptide (MDP) analogue, or combinations thereof. The adjuvant may be formulated in soluble form or in a nanoparticle, such as polylactic glycolic acid nanoparticles. A vaccine formulation comprises the adjuvant formulation and an immunogen. Methods of vaccinating an animal include delivering the vaccine formulation to the animal.

### FIELD OF THE INVENTION

The present invention relates to adjuvants comprising synthetic immune receptor ligands.

### BACKGROUND

Adjuvants are known to enhance the immune response to vaccine antigens. Adjuvants may provide the benefit of the induction of long term protection, induction of long-term immune memory, reduction of the antigen amount and/or the number of vaccine doses needed for a successful immunization, and optimization of the immune response for populations with poor responsiveness. Despite extensive efforts and numerous numbers of compounds that have been identified as immunogenic, a limited number of adjuvants are licensed and available for use in animal and human vaccines. For certain complex diseases, stimulation of cell-mediated immune responses appears to be critical, and adjuvants can be employed to optimize a desired immune response, such as the induction of cytotoxic or helper T lymphocyte responses. In addition, certain adjuvants can be used to promote antibody responses in a relevant immunoglobulin class or on mucosal surfaces.

Live-attenuated vaccines typically do not need adjuvants as they are capable of initiating innate immunity, which drives subsequent adaptive responses that lead to successful clearance of the pathogen. In light of the safety concerns associated with live vaccines, vaccine formulations are increasingly based on highly purified subunit antigens and/or antigens produced by recombinant DNA technology. However, the poor immunogenicity typical of such antigenic subunits necessitates the use of adjuvants to enhance the immune responses to the protective epitopes in the vaccine. Unlike live vaccines, current inactivated/subunit vaccines formulated with the licensed adjuvants often confer shorter duration of immunity, largely induce antibody responses, require multiple immunizations to maintain protective immunity, and trigger poor cell-mediated immunity.

Aluminum salts (alum) have been used as adjuvants and may be used to benchmark new adjuvants in terms of safety, potency and performance. Alum adjuvants act primarily to increase antibody production (Th2 response) and are therefore suitable for vaccines targeting pathogens killed primarily by antibodies, but may not be effective against intracellular pathogens or cytotoxic therapeutic vaccines. Mineral oil-in-water emulsion adjuvants (Freund's incomplete adjuvant) are generally considered too toxic to use in humans.

Various combination adjuvants consisting of at least one innate immune receptor ligand with other immunogenic compounds have been developed in recent years. Examples include AS04 (Alum plus MPLA, Expert Rev Vaccines 2011, 10, 471), polyphosphazine, CPG ODN, and a defense regulated peptide (Vaccines, 2014, 2, 297) intended to stimulate both innate and adaptive immunity (Th1 and Th2 responses). Another combination, MF59 and Pam3CSK4 has been shown to boost adaptive responses to influenza subunit vaccine through an IFN type I-independent mechanism of action (J Immunol 2012, 188, 3088).

This background information is provided merely to provide information believed to be relevant to a basic understanding of the present invention. It is not an admission that any of the foregoing is prior art against any aspect of the claimed invention.

### SUMMARY OF THE INVENTION

Generally, the present invention relates to adjuvants comprising novel receptor ligands, vaccine compositions comprising such ligands, and the use of such ligands as adjuvants. In one aspect, one compound comprises a toll-like receptor TLR-2 ligand bound to a short peptide, which is preferably a chemotactic peptide. In another aspect, one compound comprises a TLR-4 ligand comprising a synthetic lipid A analogue. In yet another aspect, another compound comprises a NOD-2 ligand. In preferred embodiments, the adjuvant comprises a combination of two or more ligands described herein.

In some embodiments, an adjuvant composition comprises a combination of at least two of a TLR-2 ligand, a TLR-4 ligand and/or a NOD-2 ligand. The combination of various synthetic innate immune TLR receptor ligands may produce robust innate and adaptive immune responses.

Other aspects of the present invention provide synthetic pathways to produce the various novel innate immune receptor ligands.

In other aspects, the present invention may also provide the formulation of the immune receptor ligand compounds into nanoparticles such as PLGA (polylactic glycolic acid) nanoparticles and may further provide means to determine the encapsulation efficiency of the formulation.

In some embodiments, the immune receptor ligands comprise 14-methyl-tetradecanoic acid as a fatty acid component of any adjuvant that carries one or more lipid chain.

In another aspect, the present invention may also provide pharmaceutical compositions comprising a pharmaceutically acceptable antigen or immunogen and any one or more of the compounds described herein, in soluble form or in an encapsulated form.

This invention also encompasses vaccines or pharmaceutical compositions, single unit dosage forms, dosing regimens and kits which comprise at least one compound or composition described herein.

### BRIEF DESCRIPTION OF THE DRAWINGS

FIGS. 1A to-1D showing increased secretion of IL-6 and TNF alpha secretion of adjuvants when formulated into nanoparticles from in vitro assays.

FIG. 2 shows graphs showing increased serum IgG1 in mice vaccinated with OVA—alone and with various adjuvant combinations.

FIG. 3 shows graphs showing increased serum IgG2a in mice vaccinated with OVA—alone and with various adjuvant combinations.

### DETAILED DESCRIPTION

The key adjuvant function is to improve immunogenicity of vaccines, particularly subunit vaccines, by improving pathogen or immunogen recognition and eliciting a response similar to the natural innate immune response. A greater understanding of the mechanism of action of adjuvants and the function of immune system encouraged the development of more rationally designed modern synthetic adjuvants which could stimulate and direct the immune system towards an effective response specific to the infection/disease.

One basis for the design of compounds described herein is the fact that microorganisms have unique pattern molecules that a host innate immune system selectively recognizes by their specific receptors, such as toll-like receptors (TLRs) on cell membrane and endosomes and nucleotide-binding oligomerization domain (NOD) proteins in cytoplasm. Upon recognition of pathogen-associated molecular patterns (PAMPs) from the microorganisms, a defense response in the host immune system is triggered through the activation of macrophages, generation of surface expression molecules on the antigen presenting cells, and the production of the cascade of various cytokines.

In some embodiments, the present invention comprises an adjuvant which comprises one or more TLR and/or NOD ligands. In some preferred embodiments, the adjuvant comprises a combination of at least two of a monophosphoryl Lipid A (MPLA) analogue, a Pam3CSK4 analogue, and a muramyldipeptide (MDP) analogue. As used herein, an “analogue” is a molecule that is structurally similar to that of another compound, but differing from in respect of one or more components, which may be an atom, a functional group, or a substructure. The different ligands may be used in different and varying molar ratios. In some embodiments, the molar ratio may vary between 0:1 to 1:0 for any two components, such as 1:1, 1:2, or 1:3 or may be a non-integer ratio. For example, a combination of three different ligands may have a molar ratio of 1:1:1, 1:2:1, 1:2:2, 1:3:1, 1:3:2, or 1:3:3 or the like.

Monophosphoryl Lipid A (TLR-4 ligand): Lipopolysaccharide (LPS) is a glycolipid found in the outer membrane of bacterial cell wall, and is known to be a strong activator of the innate immunity of a host mammal by triggering many pathophysiological events initiated by infection. LPS is generally known as “endotoxin”. LPS comprises three main regions: the 0-antigen region, the core region and the Lipid-A region (Raetz WO/05687). The Lipid-A region is a hydrophobic lipid anchor of LPS and has been shown to be responsible for the biological activities of LPS. Lipid A is the detoxified version of LPS and consists of beta-(1,6)-linked D-glucosamine disaccharide at 1-O— and 4′-O-positions. Hydroxylated and non-hydroxylated fatty acids are linked to the hydroxyl and amino groups of disaccharide to confer hydrophobicity to the Lipid A. A large number of synthetic Lipid A analogs have been prepared and structure activity relationships explored (Lien et al. 2001, Christ et al 1995, Takada and Kotani, 1989, Sato, 1995 etc.). It has been shown that the minimal structure required for toxicity was a bisphosphorated beta (1,6)-linked di-glucosamine core to which long chain fatty acids are attached (Ribi et al 1982). The nature and quantity of fatty acid chains do not appear to be so rigid in dictating the endotoxicity or immune-adjuvant activity.

Lipid A has been suggested to be a ligand for TLR-4, a pattern-recognition receptor involved in the mediation of immune responses in the host system (Kutusova et al, 2001). While removal of one phosphate group results in significant loss of toxicity without substantially affecting adjuvant activity (Werner, 1996), the TLR-4 agonistic and antagonistic properties of Lipid A are governed by the number and distribution of acyl chains in the molecule (Seydel et al, 2000 and Schromm et al, 2000).

Lipid A structures derived from Escherichia Coli (A), Salmonella strains (B), Bacteroides fragilis (C) and Porphyromonas gingivalis (D) are shown below:

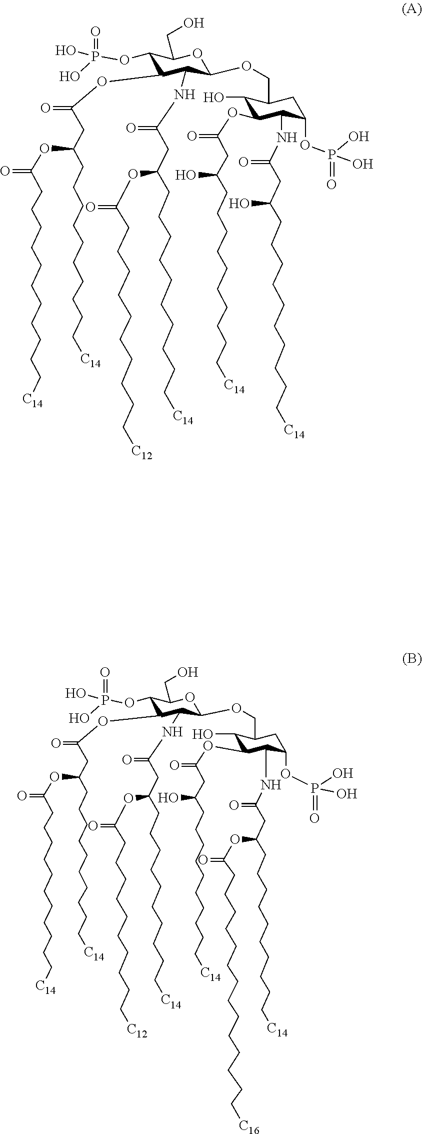

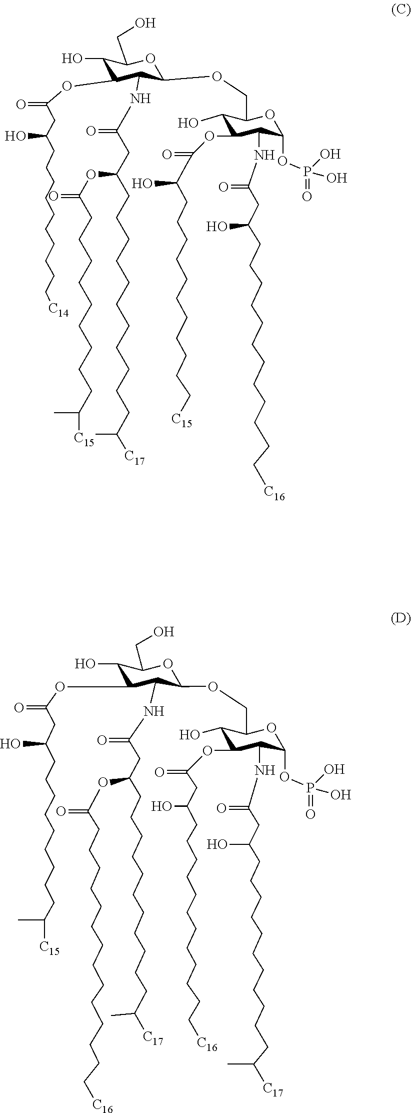

These are representative structures of the Lipid As of the particular strains and each includes a di-lipid structure. However, natural Lipid A from any source is not homogenous, the heterogeneity deriving from varying number and length of fatty acids. The nature of fatty acid lipid chains, the number of lipid chains and position of phosphate group (1- or 4′-) present on the disaccharide appears to contribute to the endotoxic activities and biological effects of the individual species or strain of the pathogen.

In some embodiments, novel structures for TLR-4 ligands are monophosphorylated and comprise at least one lipid which is a di-lipid and lipid chains of varying length, where at least one chain of the di-lipid portion is chosen to be a “fork-acid” (such as 14-methyl-tetradecanoic acid or isopentadecanoic acid) and the phosphate group is at 4′-position which provides the most common features of the structures of all the bacterial strains. A “fork acid” is a fatty acid chain which has at least one branch.

13-methyltetradecanoic acid or 15-methylhexadecanoic acid have a fork shape at the end of the lipid tail, and often appears in some bacterial strains such as Porphyromonas gingivalis strain. A single terminal methyl branch may be characterized as a terminal isopropyl unit. The small branch or fork at the end of the lipid tail on the cell wall of the bacterium may contribute to its ability to adhere to the host surface. The fork shape of the lipid tail may increase the lipophilic nature of the bacterium and strengthens its adherence to the host surface and its location on the host. For example, all Lipid-A structures from P. gingavalis contain similar branching in one or more of the lipid chains of the structures.

The incorporation of a mono or di-lipid comprising a branched lipid chain may enable the use of the adjuvant combination as a common adjuvant to be used with vaccine for many different strains of bacterial infections.

Pam3CSK4 Analogue (TLR-2 Ligand): Pam3CSK4 is a triacylated peptide, having the formula shown below, and has been shown to be recognized by TLR-2, or the heterodimer of TLR2/TLR1 or TLR4/TLR2.

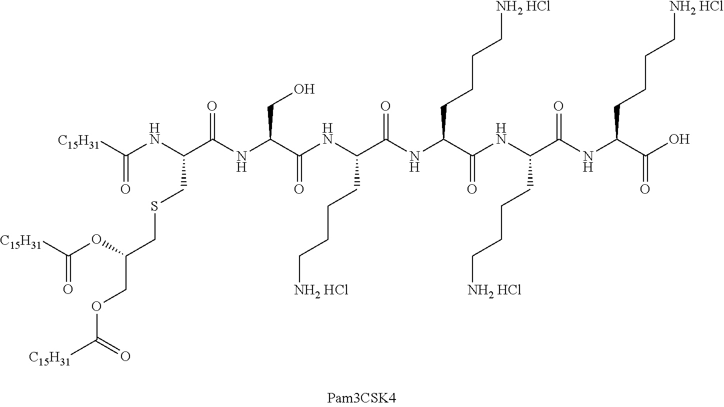

In embodiments of the present invention, novel TLR-2 ligands comprise Pam3CSK4 analogues, having different aliphatic fatty acid chains and having variations in the peptide chain. One derivative comprises a fork acid such as 14-methyltetradecanoic acid in place of a palmitic acid chain. The variations of the peptide chain include variations in the amino acid composition and/or the inclusion of a chemotactic peptide, fMLP (N-fomylated methionine-leucine-phenylalanine), in some cases.

Chemotaxis plays a critical role in the immune response, including in the directed migration of leukocytes to sites of infection and the trafficking of lymphocytes. Dysregulation of chemotactic responses has been implicated in the pathogenesis of inflammatory diseases, such as asthma and arthritis. Excessive production of the proinflammatory cytokines, TNF-alpha and IL-1beta caused during sepsis can be effectively controlled by activating formyl peptide receptors (FPRs) [J. Immunol 2010, 185, 4302-4310].

The tripeptide, N-formyl methionine-leucine-phenylalanine (N-Formyl-Met-Leu-Phe or fMLF), widely referred as fMLP, is a potent chemotactic peptide and macrophage activator. Attaching fMLP to a TLR ligand is a novel concept and the resulting compound may activate the respective TLR family receptor as well as FPR. The resulting immune responses may be better directed towards the infection rather than simply causing inflammation.

In some embodiments, fMLP may be attached to a TLR-2 ligand using a spacer, such as ethylenediamine, to allow the immune system to recognize both motifs independently. The tri-peptide, fMLP also can be attached to a TLR-4 ligand or MDP or any other immune receptor ligands to increase the immunogenicity.

Lipidated muramyldipeptides (NOD-2 ligand): Muramyl dipeptide (MDP), a peptidoglycan subunit, is an essential component of both Gram-positive and Gram-negative bacterial cell walls [J. Am. Chem. Soc. 2012, 134, 13535-13537]. Lipidated muramyldipeptides (LMDPs) are lipid attached MDPs which are relatively less hydrophilic than the original MDP molecule. They are known to exhibit adjuvant activity and antitumor potency [Med Res Rev. 1984 April-June; 4(2):111-52]. The activation of intracellular pattern recognition receptor NOD2 by agonistic LMDPs leads to the release of pro-inflammatory cytokines and cellular adhesion molecules. It appears that regardless of the mechanism of recognition of bacterial structures by the innate immune receptors, the defense mechanism of the host immune system include cytokine release and dendritic cell and macrophage activation. (Kang et al, Immunity, 2009).

### Compounds

In one aspect, the present invention comprises compounds of Formula (I).

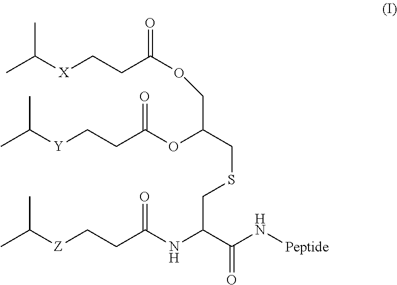

wherein X, Y, and Z are spacers, which may be the same or different, and are preferably an alkyl chain —(CH₂)ₘ— where m is an integer from 6 to 14.

The peptide comprises a linear or branched chain of 4 to 10 amino acids. In some embodiments, the amino acids are L- or D-amino acids, such as serine (Ser, S), lysine (Lys, K), phenylalanine (Phe, F), leucine (Leu, L), methionine (Met, M), asparagine (Asp, N), glutamine (Gln, Q) and alanine (Ala, A). The peptide may comprise one single peptide chain or two short peptides separated by a linker or spacer, such as a short alkyl chain.

In some embodiments, the peptide comprises a first peptide comprising 4 to 7 amino acids linked to a second peptide chain which comprises a chemotactic peptide. Preferably, the first peptide comprises Ser, Lys, Asp, Ala, Gln and Lys. In some embodiments, the first amino acid is Ser and the last amino acid is Lys. The chemotactic peptide may be a tripeptide comprised of Phe, Leu and formyl Met. The linker which separates the first and second peptides may comprise an alkyl chain, such as —(CH₂)ₙ— where n is an integer from 2 to 10.

Exemplary compounds include:

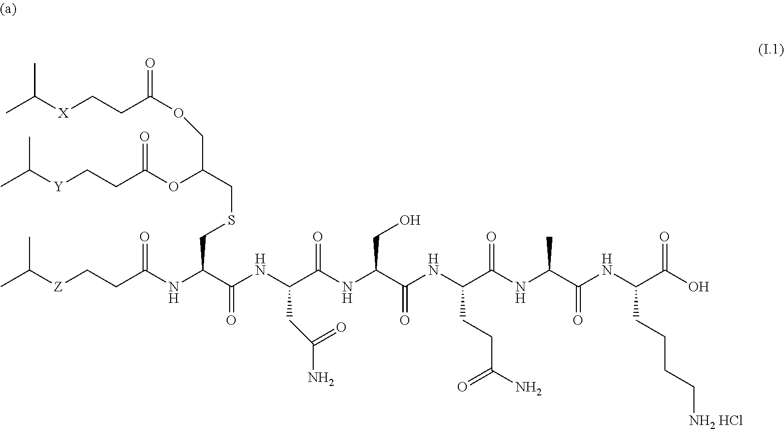

X=Y=Z=—(CH₂)ₘ— where m is an integer from 6 to 20; or

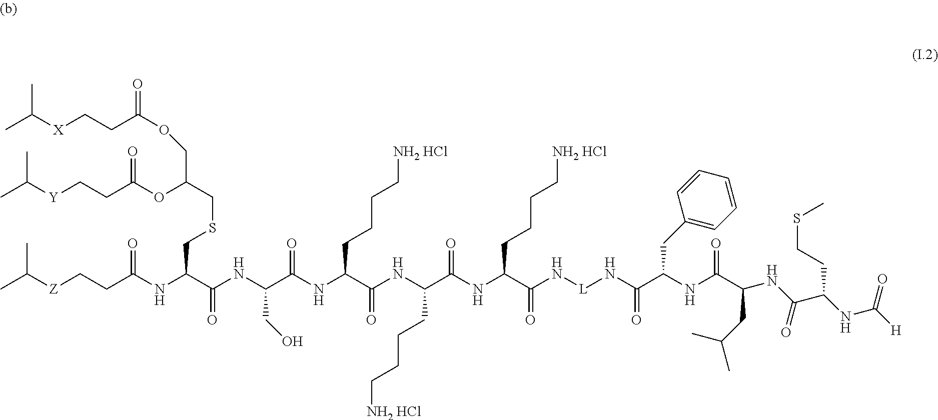

X=Y=Z=—(CH₂)ₘ— where m is an integer from 6 to 20 and L=linker=—(CH₂)ₙ— where n is an integer, from 2 to 10.

In one embodiment, the compound has the structure of Formula I.3:

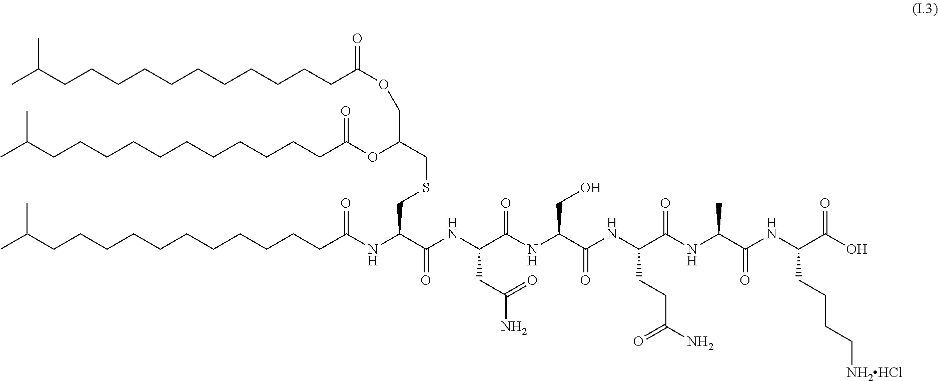

C₇₂H₁₃₄ClN₉O₁₅S, MW: 1433.40 (Compound 31, Example-1)

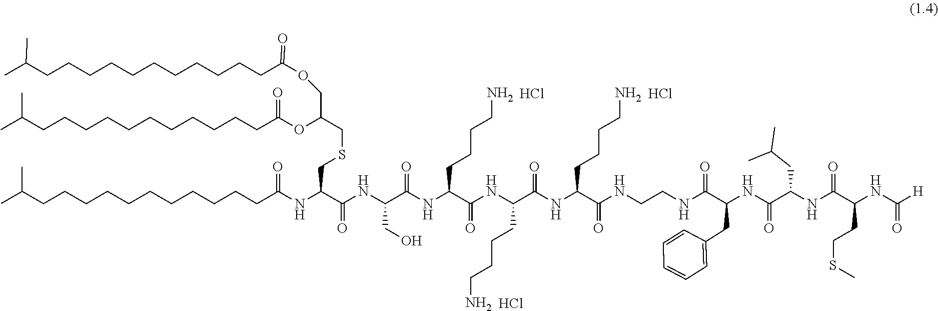 [SMILES: CSCC[C@H](NC=O)C(=O)N[C@@H](CC(C)C)C(=O)N[C@@H](Cc1ccccc1)C(=O)NCCNC(=O)[C@H](CCCCN)NC(=O)[C@H](CCCCN)NC(=O)[C@H](CCCCN)NC(=O)[C@H](CO)NC(=O)[C@H](CSCC(COC(=O)CCCCCCCCCCCC(C)C)OC(=O)CCCCCCCCCCCC(C)C)NC(=O)CCCCCCCCCCCC(C)C ; Cl ; Cl ; Cl]

C₉₅H₁₇₆Cl₃N₁₃O₁₅S₂, 1908.19 (Compound 52, Example-2)

In another aspect, the present invention may comprise compounds of Formula (II), comprising lapidated muramyldipeptide (LMDP) scaffolds. Muramic acid is a major building block of the bacterial cell wall and comprises lactic acid joined to a glucosamine ring by an ether linkage.

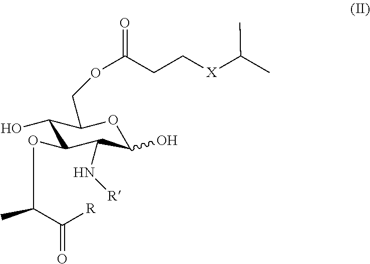

wherein X is a linker.

In some embodiments, X is an aliphatic chain, such as —(CH₂)ₘ— where m is an integer from 6 to 14; R′ is either an acetyl or a glycolic group; and/or R is a short peptide comprised of L- and D-amino acids, such as a dipeptide or tripeptide comprised of Ala, isoglutamine (IsoGln) and/or Ser. The aliphatic chain may comprise a medium or long chain fatty acid moiety. In one embodiment, the fatty acid has a chain length of 6 to 20 carbon atoms. Preferably, the fatty acid comprises a terminal isopropyl unit.

One example comprises a compound of Formula II.1 or Formula II.2 (where X=—(CH₂)₉₎

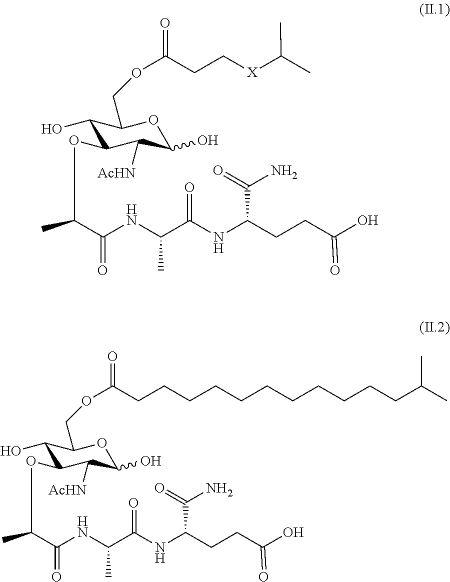

C₃₄H₆₀N₄O₁₂, MW: 716.86 (Compound 63, Example-3)

In another aspect, the present invention comprises a TLR-4 ligand, which may be a compound of Formula (III):

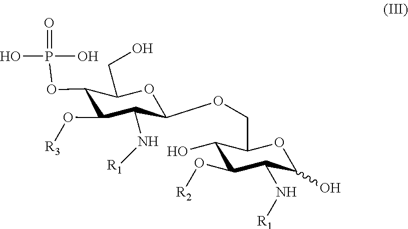

wherein R1 is a di-lipid, R2 is a medium or long chain fatty acid group, and R3 is a medium or long chain fatty acid or a di-lipid.

In some embodiments, R1 is a di-lipid having the structure shown in formula III.1 or III.2:

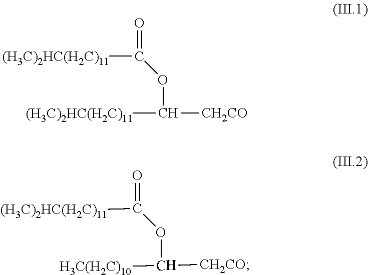

R2 is a palmitoyl group, having the formula, CH3-(CH2)₁₄—CO; and
R3 is either a palmitoyl group or a lipid of Formula III.3:

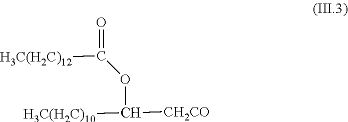

In one embodiment, the compound has the structure of formula III.4 (where R1 has the formula III.1 and R2 and R3 are palmitoyl groups):

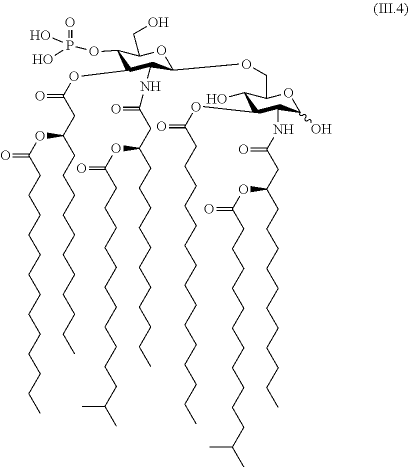

C₁₁₄H₂₁₅N₂O₂₂P; MW: 1996.90 (Compound 94, Example-4)

In another embodiment, the compound has the structure of formula III.5

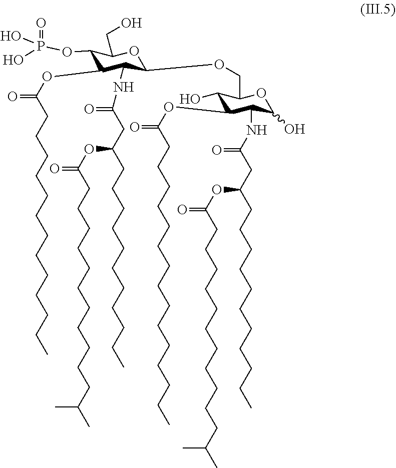

C₁₀₂H₁₉₃N₂O₂₀P; MW: 1797.39 (Compound 95, Example-5)

In yet another embodiment, the compound has the structure of formula III.6

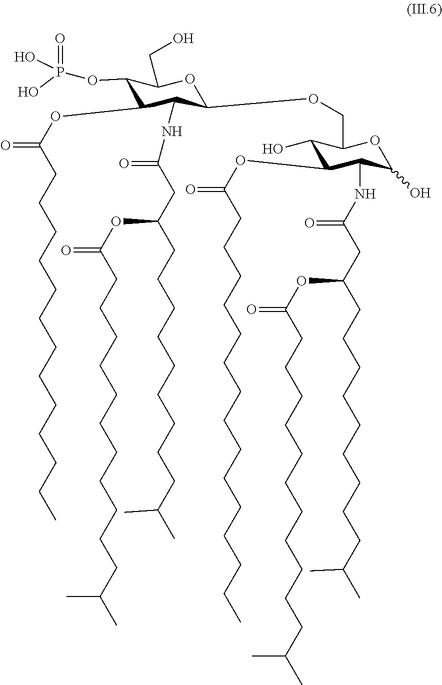

C₁₀₈H₂₀₅N₂O₂₀P; MW: 1882.76 (Compound 96, Example-6)

In some embodiments, the lipid A analogues described herein may comprise, or be used in combination with, known lipid A analogues, such as a compound commonly known as 7-acyl lipid A which has the structure of Formula III.7:

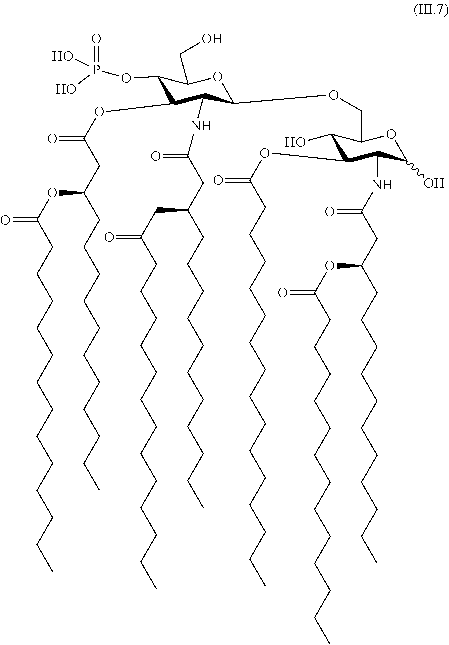

C₁₁₀H₂₀₇N₂O₂₃P; MW: 1956.80 (Compound 97, Example-7)

Embodiments of the invention further include within its scope a racemic mixture, stereoisomer, an enantiomer, a tautomer or a pharmaceutically acceptable salt of any compound described or claimed herein. The pharmaceutically acceptable salts include the conventional non-toxic salts or the quaternary ammonium salts of the parent compound derived from inorganic acids such as hydrochloric, sulfamic, phosphoric, and nitric; and from organic acids such as acetic, succinic, glycolic, stearic, lactic, malic, tartaric, citric, ascorbic, maleic, glutamic, salicylic, sulfanilic, fumaric, toluenesulfonic, methanesulfonic and oxalic and the like.

### Methods of Preparation

The compounds described herein can be synthesized using the methods described below, or similar methods, together with synthetic methods known in the art of synthetic organic chemistry, or by variations thereon as appreciated by those skilled in the art. Preferred methods may include, but are not limited to, those described below. The reactions are performed in a solvent or solvent mixture appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

Protection and de-protection in the processes below may be carried out by procedures generally known in the art (see, for example, Greene, T. W. et al, Protecting Groups in Organic Synthesis, 3rd Edition, Wiley (1999)). General methods of organic synthesis and functional group transformations are found in: Trost, B. M. et al, eds., Comprehensive Organic Synthesis: Selectivity, Strategy & Efficiency in Modern Organic Chemistry, 1st Edition, Pergamon Press, New York, N.Y. (1991); March, J., Advanced Organic Chemistry.

Some compounds described herein may be prepared using the synthetic scheme as outlined in Scheme 1. The reaction conditions such as temperature, time, choice of solvent and workup procedures are selected which may be suitable for experimental conditions recognized by one skilled in the art. Restrictions to the substituents that are compatible with the reaction conditions will be readily apparent to one skilled in the art and alternate or analogous methods must then be used.

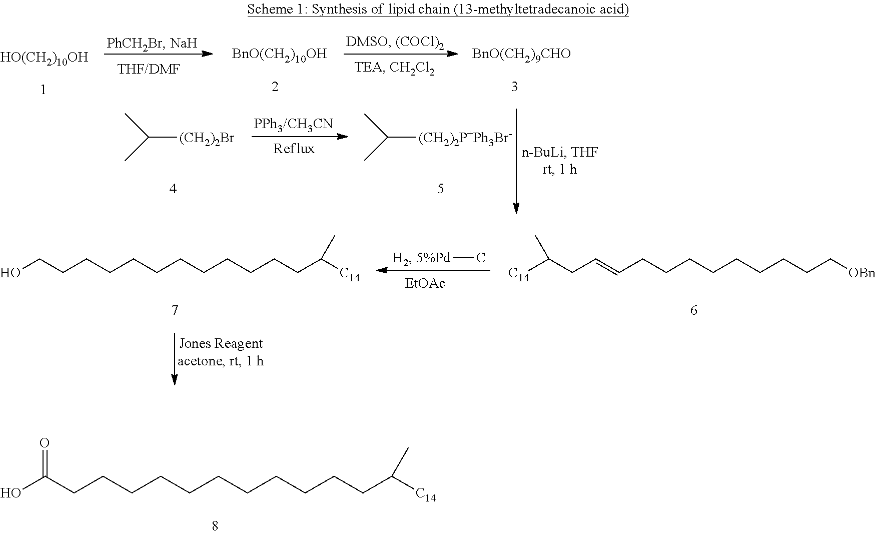

One of the two hydroxyl groups of decane-1,10-diol was selectively protected and the second hydroxyl group was oxidized to aldehyde by Swern Oxidation. Wittig reaction with isoamyltriphenylphosphorylbromide ylide followed by hydrogenation provided 13-methyltetradecanol which upon Jones oxidation provided 13-methyltetradecanoic acid.

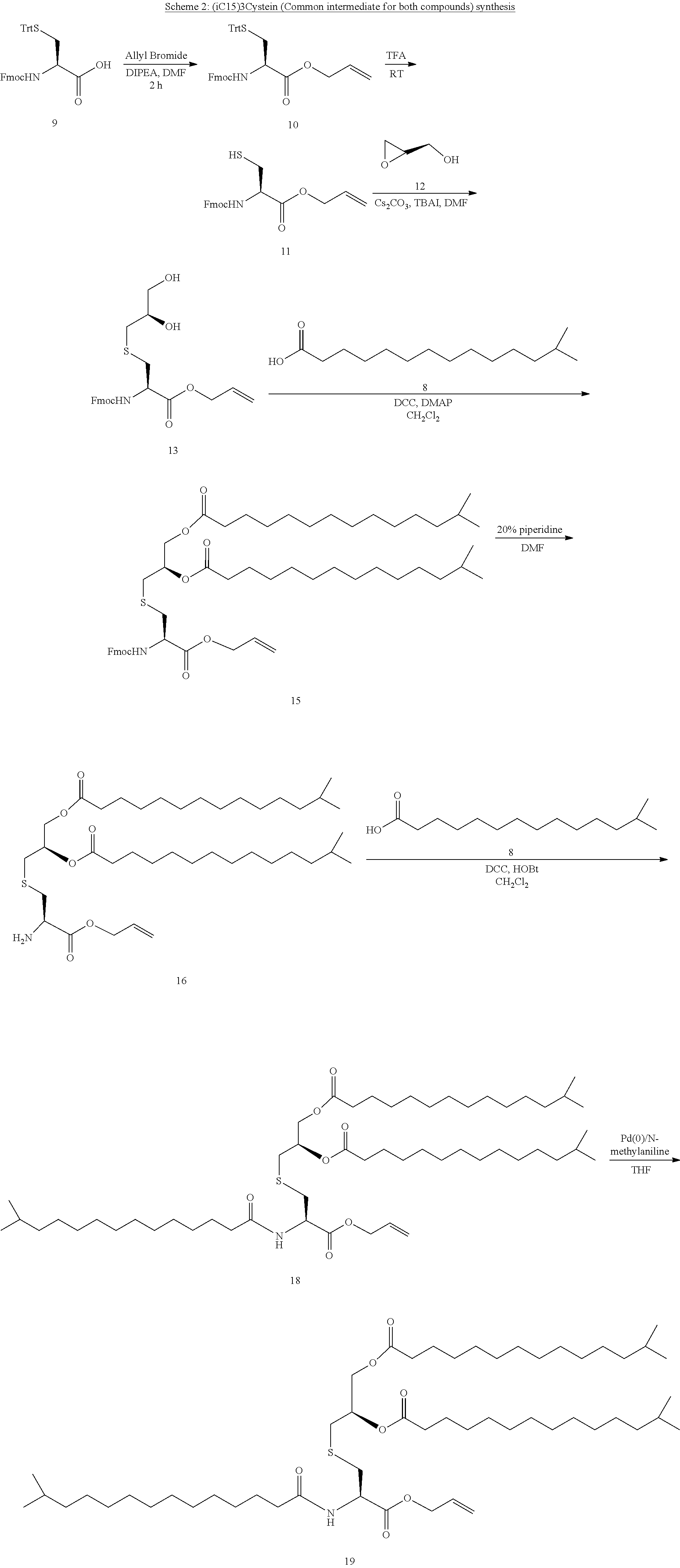

The acid group of commercially available trityl protected Fmoc-Cystein was protected with allyl and then trityl was removed. Coupling with commercially available R-glycidol provided the dihydroxy compound which upon esterification of both hydroxyl groups with 13-methyltetraadecanoic acid (iC15) provided the dilipocystein. Fmoc was removed and the third lipid was introduced through peptide coupling followed by removal of allyl protection provided the triplipocystein free acid (iC15)3C—OH.

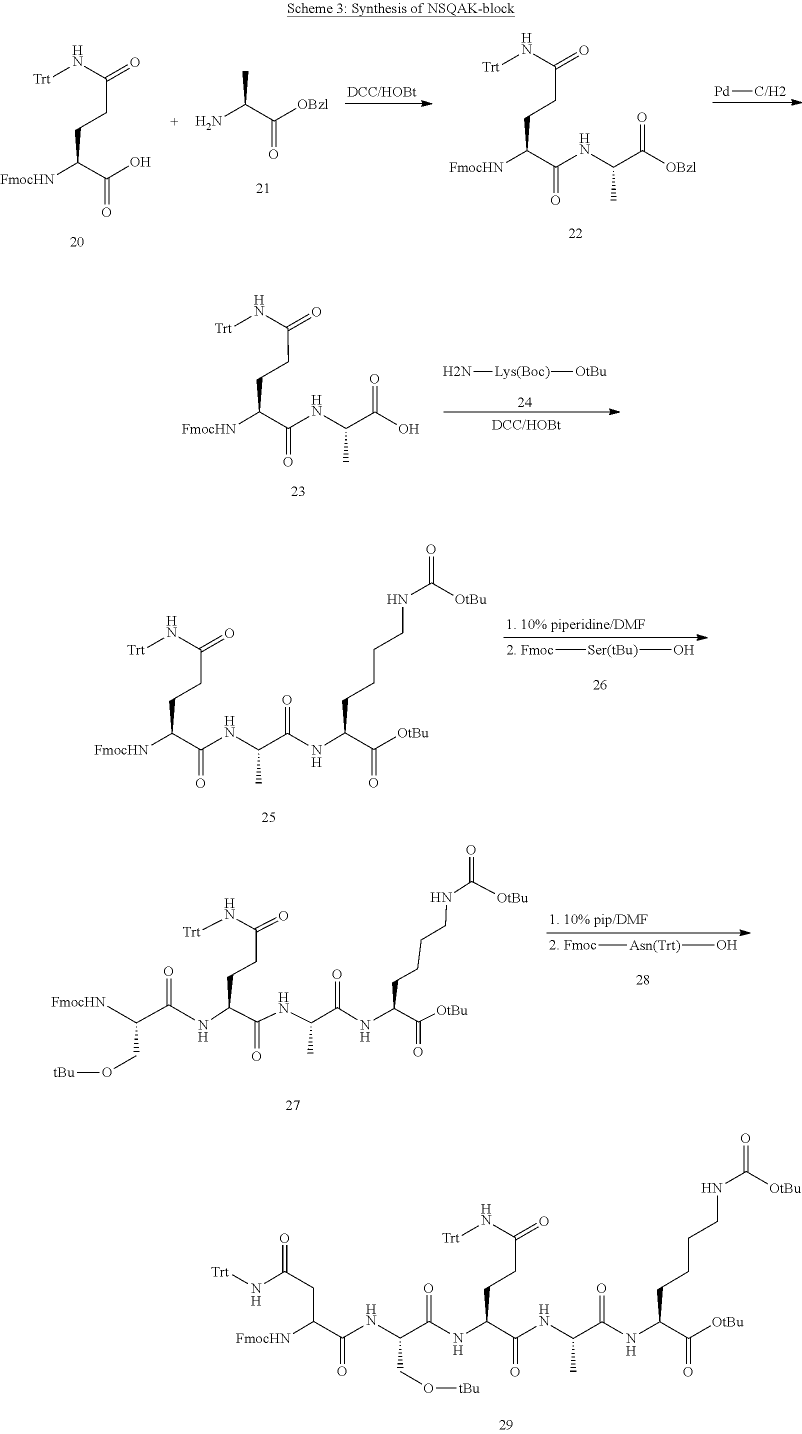

The pentapeptide building block with all acid sensitive protecting groups was prepared starting from properly protected L-alanine and L-lysine using solution phase Fmoc chemistry with a sequential coupling of L-glutamine, L-serine and L-asparagine.

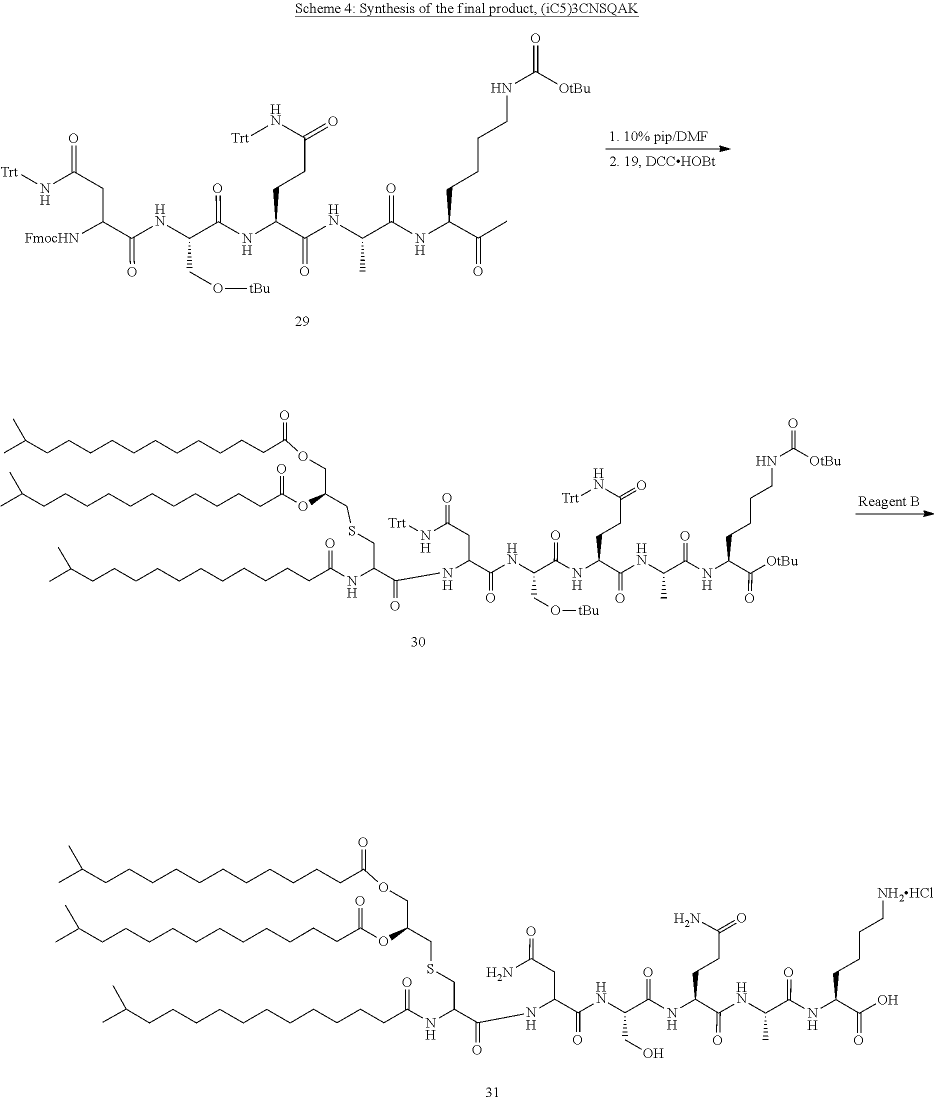

The trilipocystein free acid (iC15)3C—OH, was coupled to the Fmoc deblocked pentapeptide using DCC/HOBt method and the product was upon treating with TFA provided the final product which was purified by column chromatography followed by lyophilization with 0.1NHCl to isolate the compound as HCl salt.

B. (iC15)3CSK3-eda-FLMf

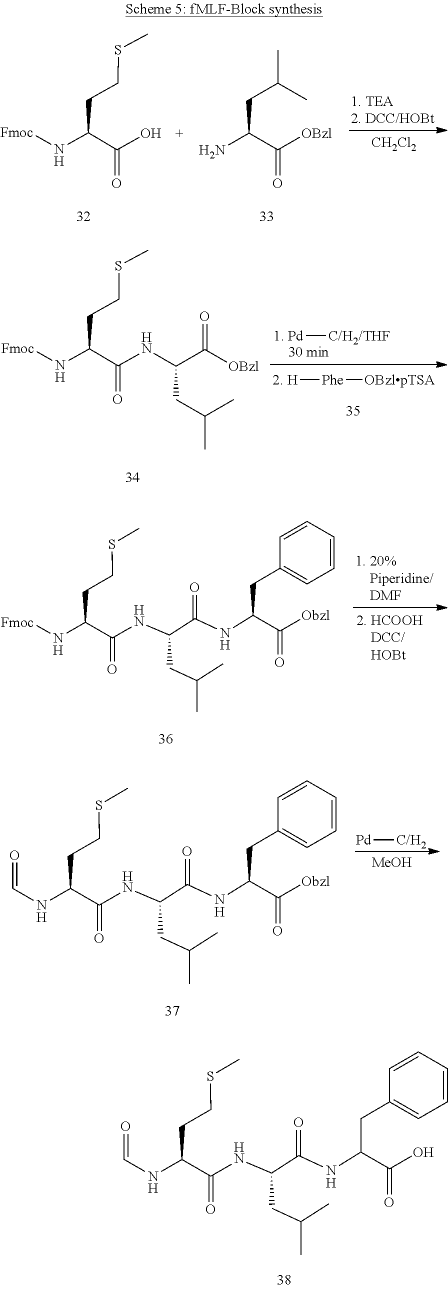

This tripeptide was synthesized starting from Fmoc-protected methionine, leucinebenzylester and phenylalaninebenzyl ester. Upon formylation of the amine end of the tri-peptide, the benzyl protection of phenylalanine was removed to give the free acid.

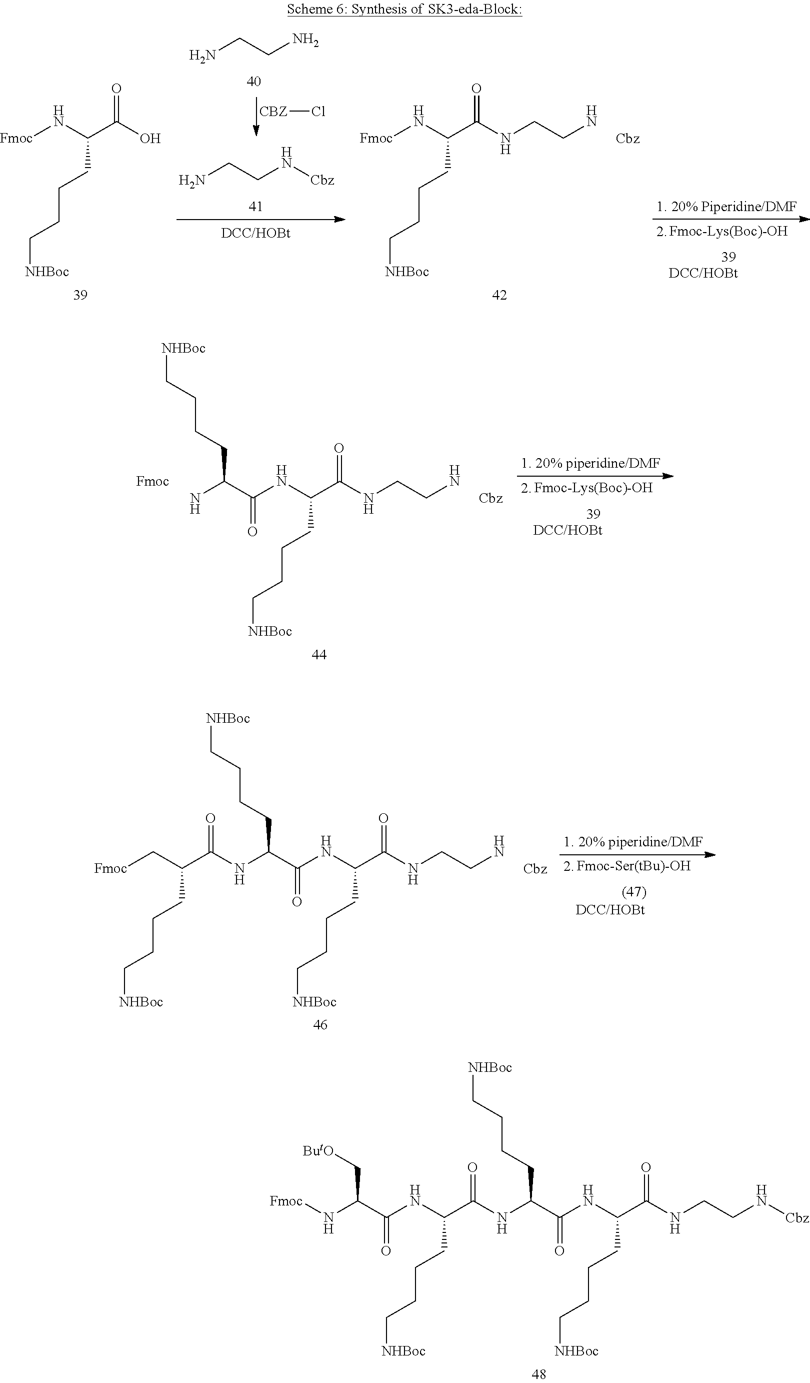

Fmoc-Lysine was coupled to the spacer, ethylenediamine and the chain was extended from the amine end of the lysine with sequential coupling of lysines and serine.

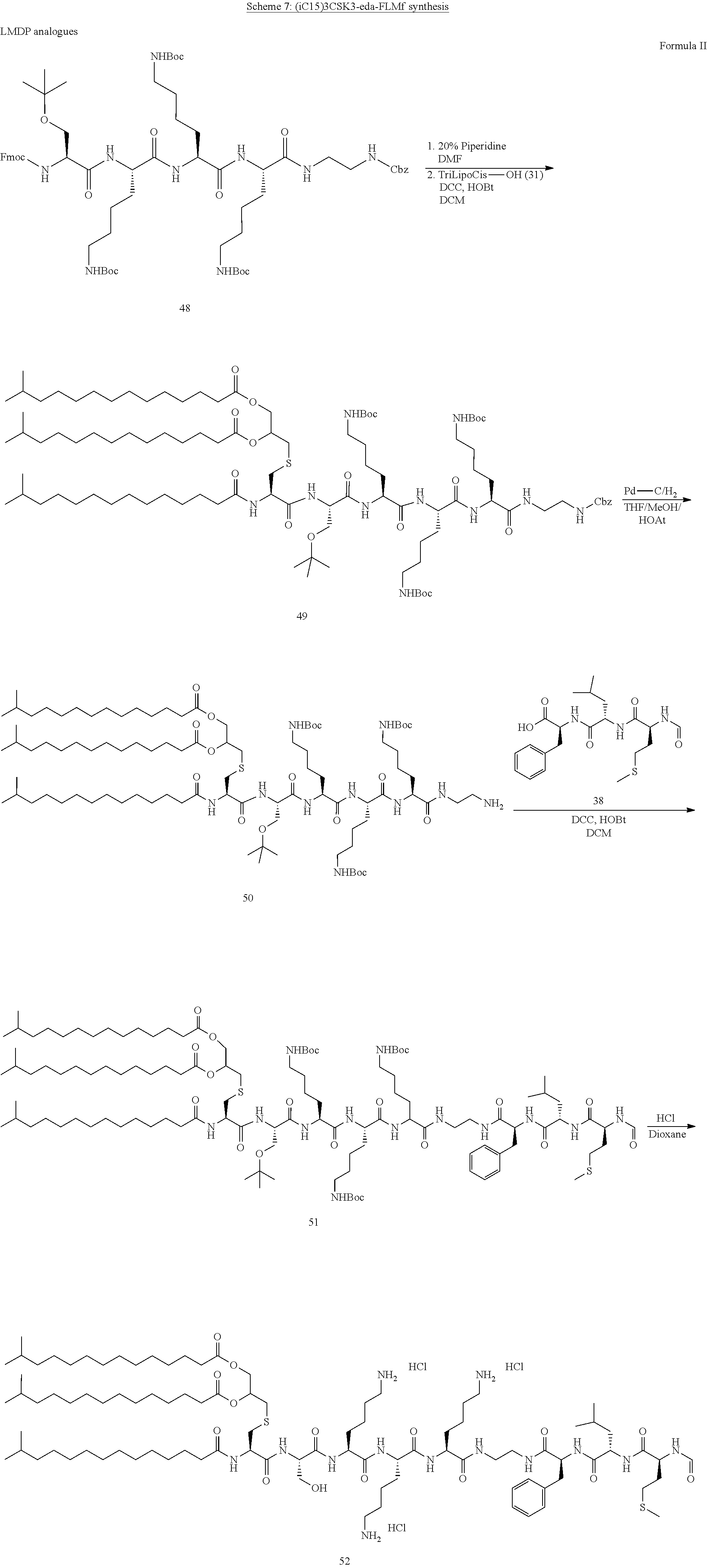

Fmoc protection on the Serine end of SK3-eda-block was removed using standard conditions and coupled to TrilipoCys-OH block and then Cbz protection was removed to couple to fMLF-block. The final protected compound was treated with Dioxane-HCl to give the final product as HCl salt.

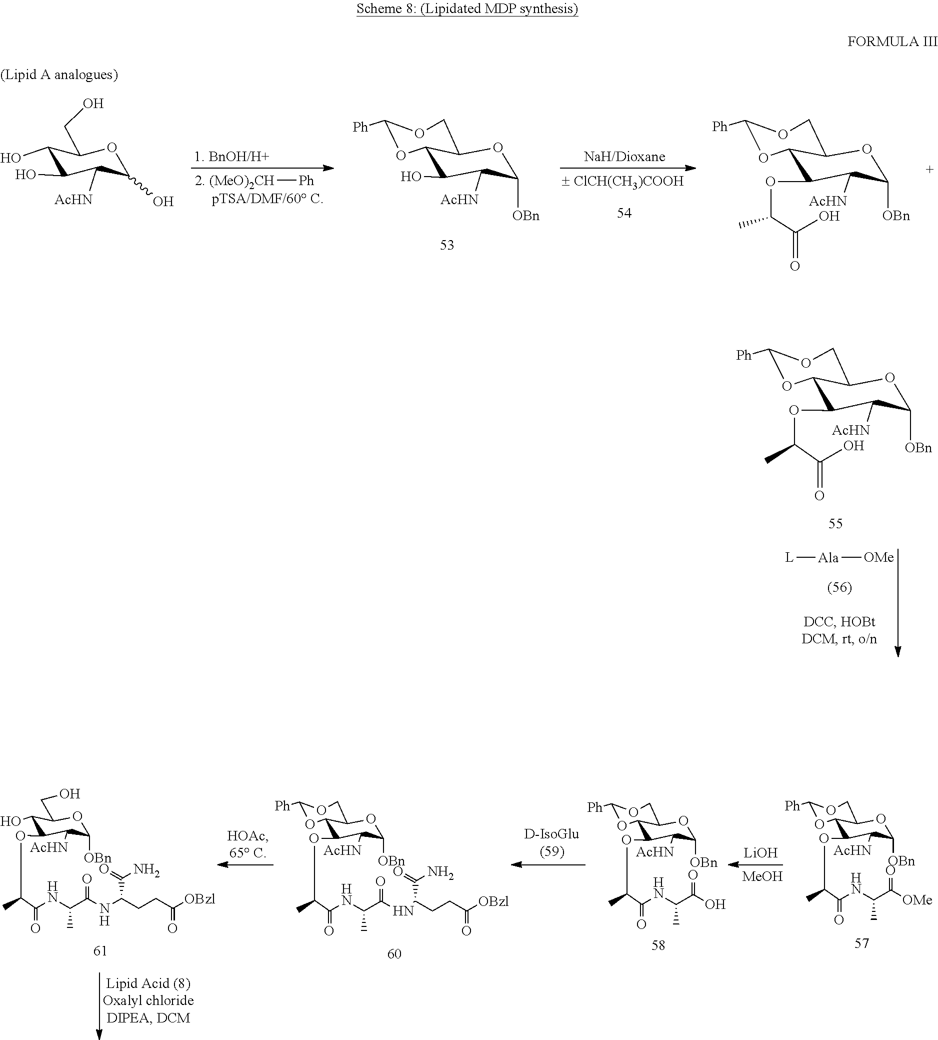

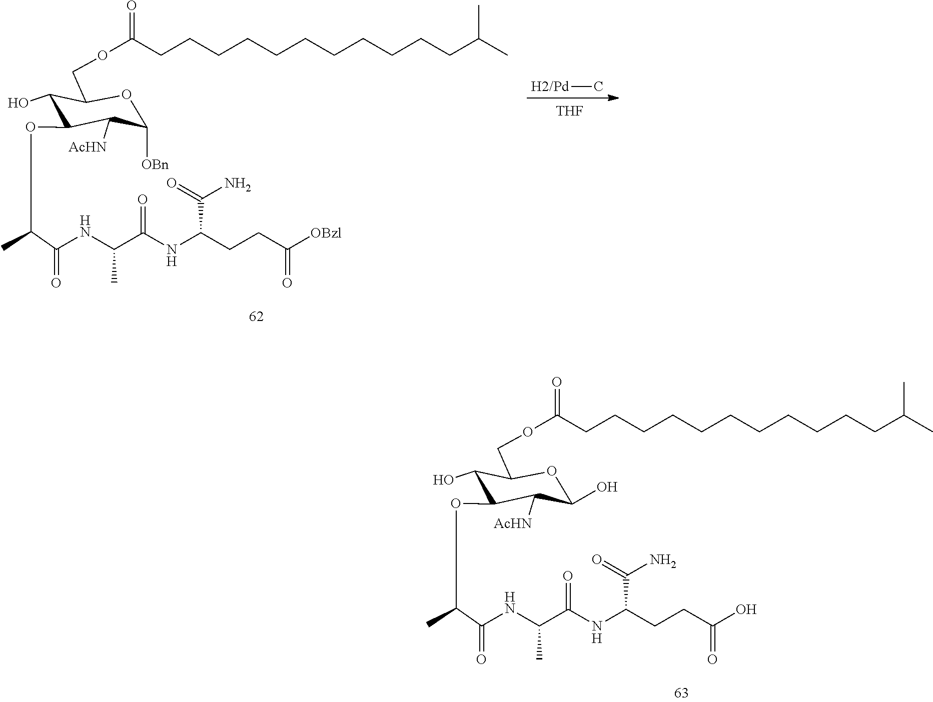

4,6-hydroxy groups of N-Acetyl-2-deoxy-D-Glucose were first protected with benzylidine group and the 3-hydroxy was coupled with racemic 2-chloropriopionic acid to give the diastereomeric mixture of muraminic acids which can be separated by column chromatography. The compound with little other isomer was carried forward to couple with aminoacids such as alanine and D-Isoglutamine.

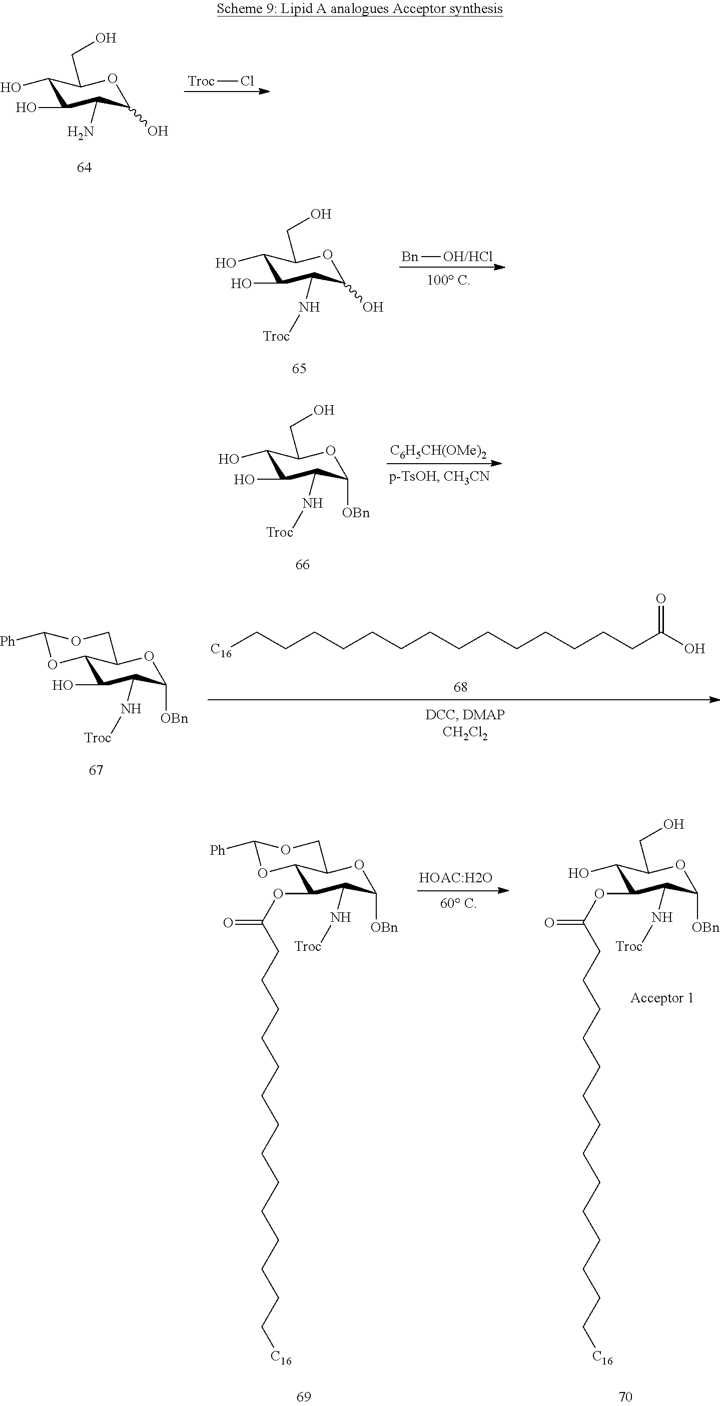

Troc protection was installed on amino group of D-glucosamine.HCl first and the anomeric hydroxyl was protected with benzyl group. 4- and 6-hydroxy groups were protected with benzylidine protection which was removed after palmitic acid coupling at 3-position.

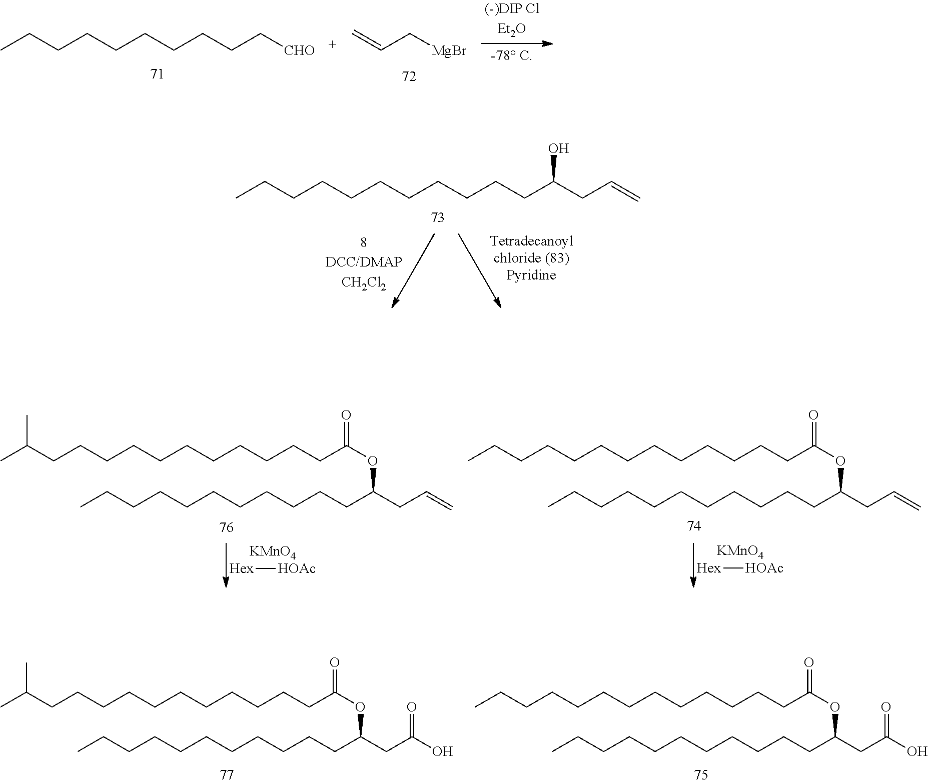

Three dilipid acids were synthesized using a common procedure. (R)-3-(13-methyltetradecanoyloxy)tetradecanoic acid (77) or (R)-3-(tetradecanoyloxy)tetradecanoic acid (75) were synthesized using Scheme 10 and (R)-15-methyl-3-(13-methyltetradecanoyloxy)hexadecanoic acid, (81) was synthesized using Scheme 10A.

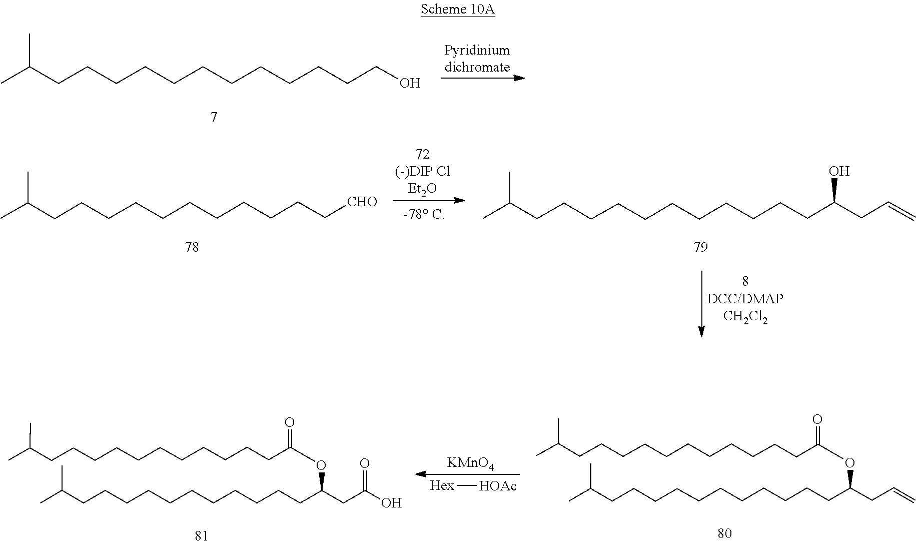

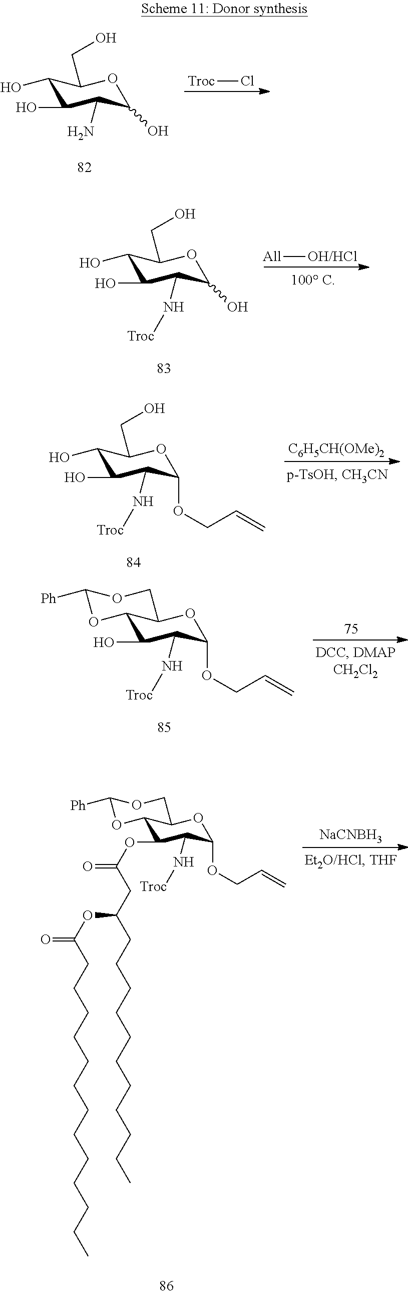

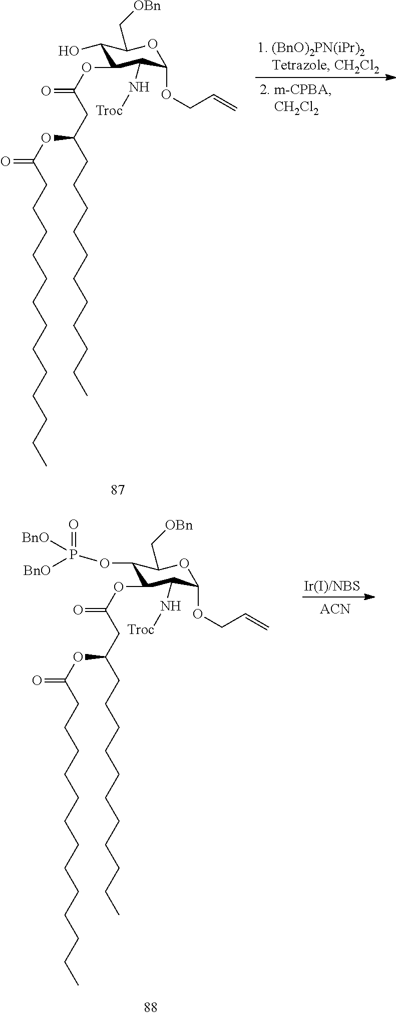

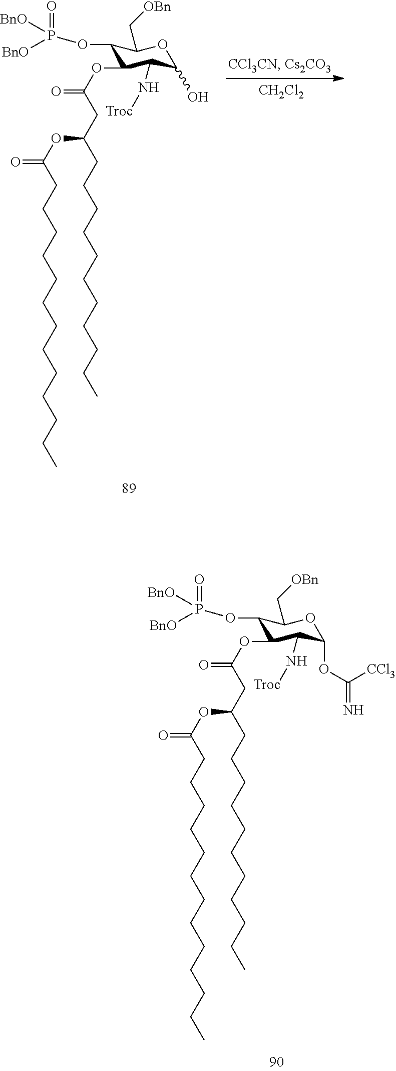

Glucosamine hydrochloride was modified to have Troc protection on amine at 2-position, protected phosphate at 4-position and benzyl at 6-position. Lipid acid, either palmitic acid (68) or the dilipid acid (75) were introduced at 3-position a shown in Schemes 11. Regioselctive benzylidene-ring opening with sodium cyanoborohydride with ethereal HCl solution afforded the 4-OH compound, 87. The phosphate group introduction at 4-OH position was achieved by reacting with dibenzyl diisopropylphosphoramidite to form phosphate, followed by oxidation with meta-chloroperbenzoic acid (mCPBA) provided compound 88 in 77% yield. Deprotection of allyl using iridium complex followed by NBS oxidation was carried out smoothly to afford 1-hydroxy compound, 89 which was converted into desired trichloroacetamidate donor using trichloroacetonitrile and cesium carbonate as a base in 80% yield.

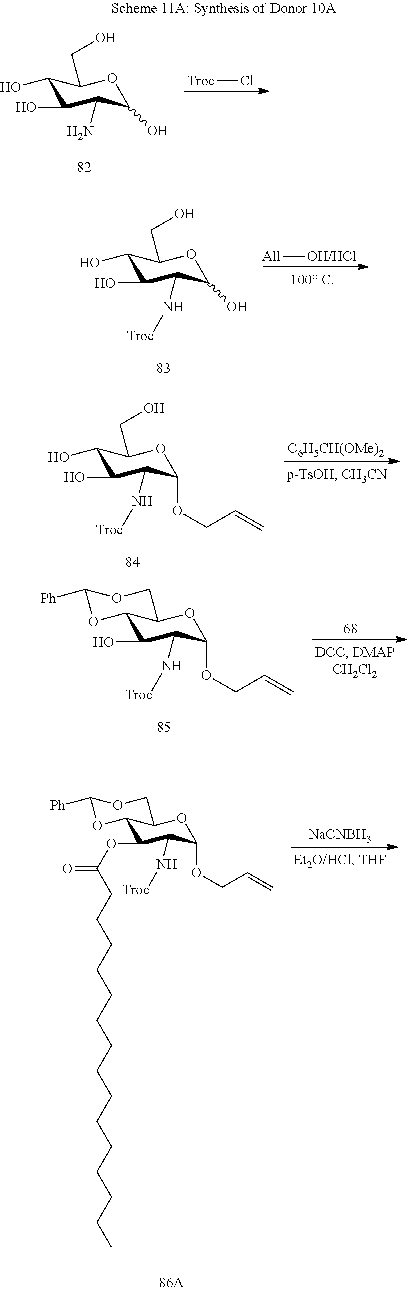

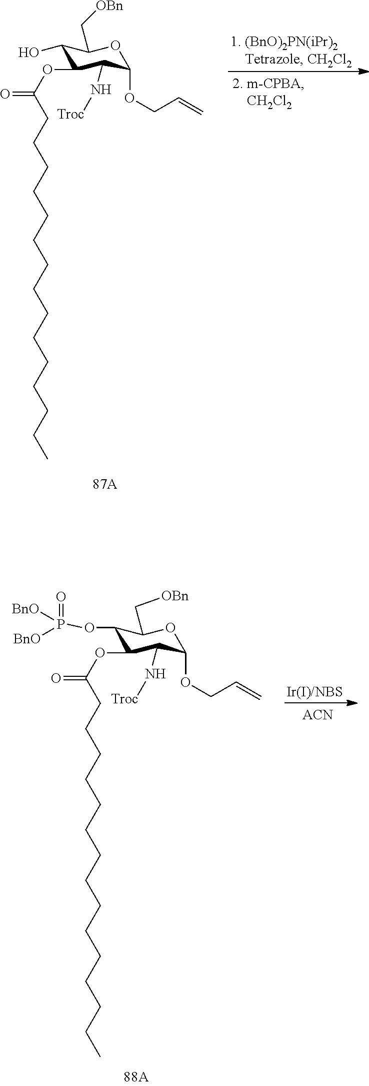

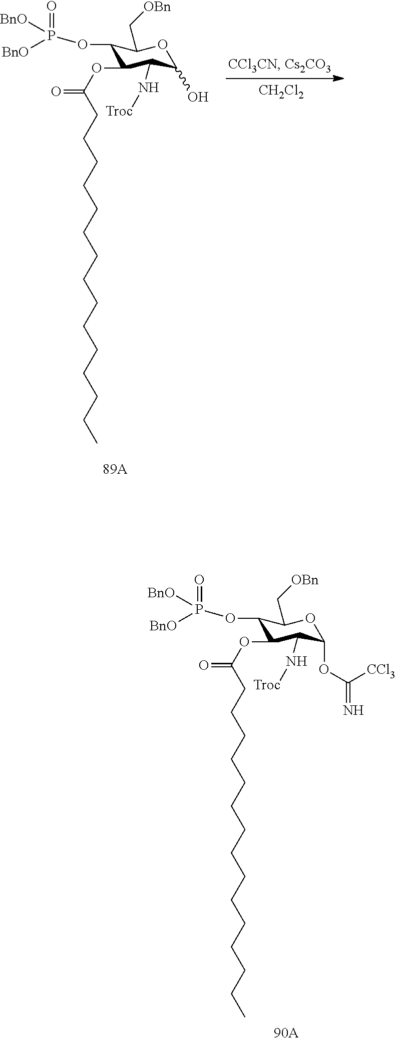

Donor, 90A was synthesized by following the same strategy as described for Scheme 11, using palmitic acid, 68 in place of the dilipid acid, 75.

### Final Assembly of the Lipid a Structures:

Glycosylation reaction between the donor, 90/90A and the acceptor 70 was achieved at room temperature by activating with trimethylsilyltriflate catalyst. The glycosylation was selective to 6-position so there was no need to protect the 4-hydroxyl group. Then the troc group was removed from the disaccharide, 91/91A by stirring with zinc powder in acetic acid to provide the diamine, 92/92A which was then treated with fork lipid acid, 77 or 80 at 2- and 2′-positions at the same time. Standard hydrogenation reaction afforded the corresponding final Lipid A derivative, 94/95/96 as a fluffy white solid after silica gel column purification followed by lyophilization in tert.butanol.

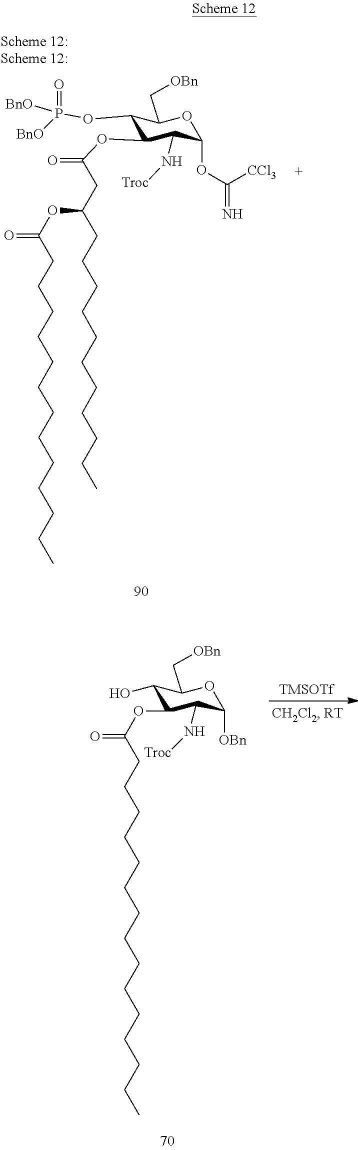

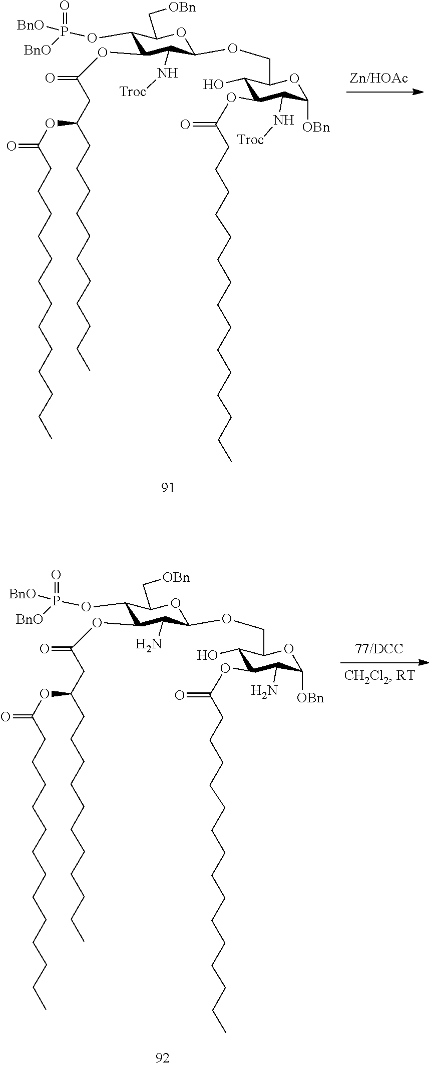

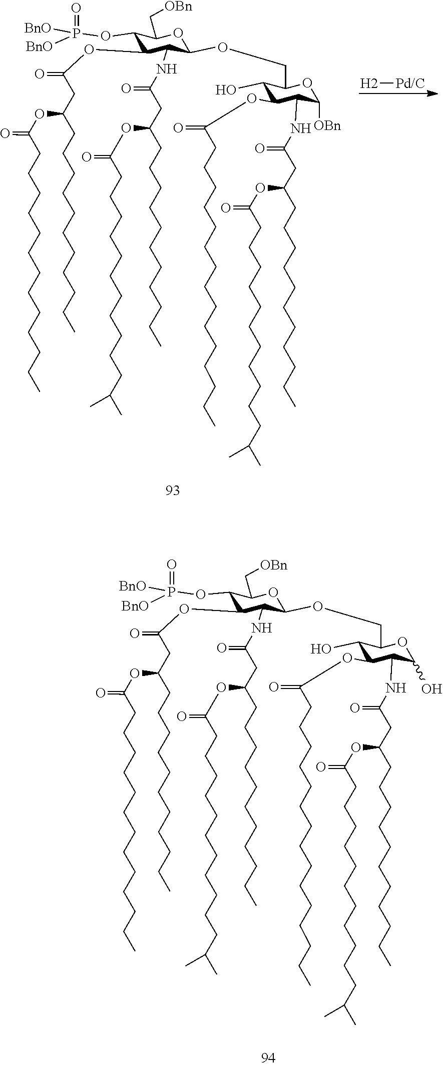

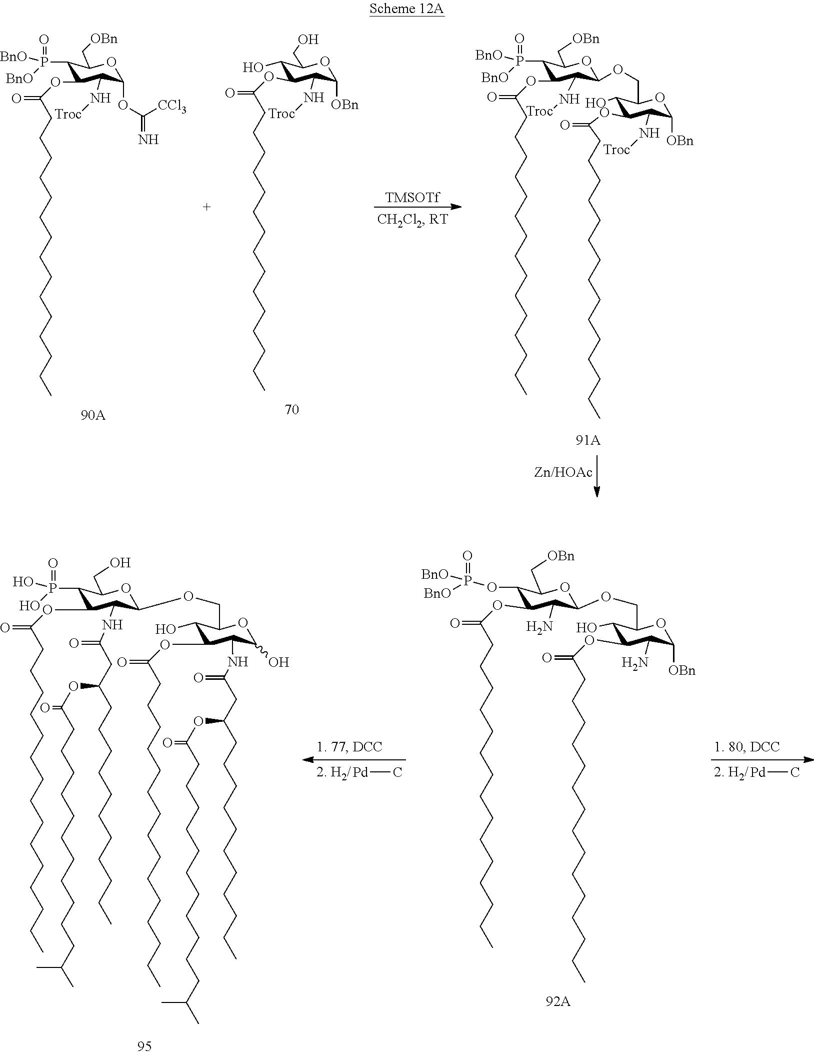

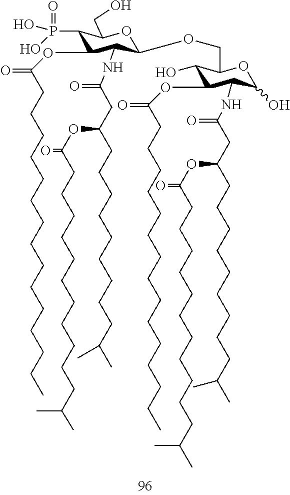

### Formulations and Compositions

Some or all of the compounds of the present invention may be formulated in soluble form or solution, or with nanoparticles, such as with a copolymer of polylactide and glycolic acid known as polylactide-co-glycolide. Such nanoparticle formulations are well-known in the art and need not be described further here. Polylactic-co-glycolic acid (PLGA) nanoparticles are known for their ability to target dendritic cells and promote their maturation, trafficking and interaction with T-cells. Dendritic cells (DCs) are known to be highly effective in antigen processing and presentation, linking innate and adaptive immunity, through a TLR specific pathway (J. Samuel, J Drug Targeting, 11, 495-507, 2003). PLGA nanoparticles are biodegradable into their monomers, lactic acid and glycolic acid, which are native to humans and hence the nanoparticle formulation offers sustained release of the innate ligands and provide robust responses with small doses of ligands.

In other aspects, the invention may comprise a pharmaceutically acceptable composition comprising at least one immunogen and at least one adjuvant ligand described herein, and preferably a combination of two or more adjuvant ligands. The pharmaceutically acceptable composition, when administered to a subject, can elicit an immune response against a cell or virus that expresses the antigen. The pharmaceutically acceptable compositions of the present invention can be useful as vaccine compositions for prophylactic or therapeutic treatment of any disease or symptoms thereof, including infectious diseases or neoplastic diseases.

For example, in some embodiments, the vaccine immunogen can include one or more immunogenic peptides, a composition or pool, of immunogenic peptides, a fusion fragment or a fusion polypeptide. In other embodiments, a fusion fragment or fusion polypeptide can be produced, for example, by recombinant techniques or by the use of appropriate linkers for fusing previously prepared polypeptides or fragments.

The suitable dosage of an immunogen described herein will depend upon the age, sex, health, and weight of the subject in need of treatment, the kind of concurrent treatment, if any, the frequency of treatment, and the nature of the effect desired. However, the most preferred dosage can be tailored to the individual subject, as determined by the skilled artisan. The total dose required for any given treatment may commonly be determined with respect to a standard reference dose (e.g., as set by a manufacturer), such as is commonly done with vaccines, such dose being administered either in a single treatment or in a series of doses, the success of which will depend on the production of a desired immunological result (i.e., successful production of a humoral and/or CTL-mediated response to the antigen(s), which response gives rise to an immune response to a cell that expresses the immunogen). Thus, the overall administration schedule must be considered in determining the success of a course of treatment and not whether a single dose, given in isolation, would or would not produce the desired immunologically therapeutic result or effect.

The therapeutically effective amount of a composition containing one or more of immunogens, is an amount sufficient to elicit an effective humoral and/or CTL response to inhibit, reduce, and/or arrest progression of cells that express the immunogen. Thus, this dose will depend, among other things, on the identity of the immunogen(s) used, the nature and severity of the subject's condition, the extent of any need to prevent such a condition where it has not already been detected, the manner of administration dictated by the situation requiring such administration, the weight and state of health of the subject receiving such administration.

For such purposes, the immunogenic compositions described herein can be used against the condition or disease (e.g., cancer) by administration to the subject in need thereof by a variety of routes. The compositions can be administered parenterally or orally, and, if parenterally, either systemically or topically. Parenteral routes include subcutaneous, intravenous, intradermal, intramuscular, intraperitoneal, intranasal, transdermal, or buccal routes. One or more such routes can be employed. Parenteral administration can be, for example, by bolus injection or by gradual perfusion over time.

The compositions and/or vaccines can be prepared as injectables, in the form of aqueous solutions or suspensions. Vaccines in an oil base are also well known such as for inhaling. Solid forms that are dissolved or suspended prior to use can also be formulated. Pharmaceutical carriers, diluents and excipients are generally added that are compatible with the active ingredients and acceptable for pharmaceutical use. Examples of such carriers include, but are not limited to, water, saline solutions, dextrose, or glycerol.

Combinations of carriers can also be used. These compositions can be sterilized by conventional, well known sterilization techniques including sterile filtration. The resulting solutions can be packaged for use as is, or the aqueous solutions can be lyophilized, the lyophilized preparation being combined with sterile water before administration. Vaccine compositions can further incorporate additional substances to stabilize pH, or to function as adjuvants, wetting agents, or emulsifying agents, which can serve to improve the effectiveness of the vaccine.

In some embodiments, the pharmaceutically acceptable composition further comprises a physiologically acceptable carrier, diluent, or excipient. Techniques for formulating and administering also can be found in Remington's Pharmaceutical Sciences, Mack Publishing Co., Easton, Pa., latest edition. Pharmaceutically acceptable carriers known in the art include, but are not limited to, sterile water, saline, glucose, dextrose, or buffered solutions. Agents such as diluents, stabilizers (e.g., sugars and amino acids), preservatives, wetting agents, emulsifying agents, pH buffering agents, additives that enhance viscosity, and the like. Preferably, the medium or carrier will produce minimal or no adverse effects.

### Examples

The present invention may be described with reference to the following Examples. These Examples are provided for the purpose of illustration only.

General: Melting points were not corrected. All air and moisture sensitive reactions were performed under nitrogen atmosphere. All dry solvents including anhydrous THF, DMF and dichloromethane were purchased from Sigma Aldrich. Isocyanates were purchased from CombiBlocks. ACS grade solvents were purchased from Fisher and Caledon and used for work-up and column chromatography without distillation. TLC plates (silica gel 60 F254, thickness 0.25 mm, Merck) were purchased from VWR and visualized under UV light as well as stains such as CAM (cerium sulfate-ammonium molybdate) solution, KMnO₄ (potassium permanganate) solution, PMA (phosphomolybdic acid) solution and ninhydrine solution. Flash silica gel 60 was purchased from Silicycle, Canada. All compounds were characterized by 1H NMR and ESMS. NMRs were recorded on 400 Varian 400 MHz spectrometers with TMS as internal standard for proton chemical shifts. Electron-spray mass spectrometric analyses were performed Agilent LCMS spectrometer.

Compound 8 (13-methyltetradecanoic acid) was synthesized as per the literature procedure (Tetrahedron 54 (1998) 15701-15710).

Preparation of Compound 10 (Fmoc-L-Cys(Trt)-OAll ester): Allyl bromide (1.74 mL, 20 mmol) was added to the solution of Fmoc-L-Cys(Trt)-OH, 9 (5.86 g, 10 mmol) and potassium carbonate (1.38 g, 10 mmol) in DMF and was stirred for 2h at room temperature. Upon completion of the reaction, the reaction mixture was diluted with ethyl acetate (100 mL) and washed with water (50 mL×3) and brine (50 mL). The organic layer was dried with sodium sulfate and evaporated. The crude was then purified by silica gel column chromatography using 10-30% ethyl acetate in hexanes to afford the allyl ester, 10 as white fluffy solid (6.0 g, 96%). The analytical data is in accordance with the literature data. ES-MS (m/z) calculated for C₄₀H₃₅NO₄S: 625.23. Found 626.8 (M+H), 1H NMR (400 MHz, CDCl3): δ 7.80 (d, 2H), 7.63 (d, 2H), 7.39-7.42 (m, 10H), 7.20-7.38 (m, 10H), 5.80-5.98 (m, 1H), 5.20-5.38 (m, 4H), 4.60-4.70 (m, 3H), 4.38-4.42 (m, 4H), 4.25 (t, 2H), 2.62-2.80 (m, 2H).

Preparation of compound 11 (Fmoc-L-Cys-OAll ester): The cysteine allyl ester 10 (4.3 g, 6.87 mmol) was dissolved in 50% TFA in dichloromethane and triisopropyl silane (4.64 mL, 22.67 mmol) was added solowyl using a syringe. The reaction mixture was stirred at room temperature for one hour and then evaporated with toluene three times. The white solid thus obtained was then stirred in hexanes (50 mL) for one hour. The solid was filtered and washed with hexanes. The product, compound 11 thus obtained was then dried and used in the next step with out further purification. Yield: 2.3 g, 98%. ES-MS (m/z) calculated for C₂₁H₂₁NO₄S: 383.12. Found 384.5 (M+H).

Preparation of compound 13 ((5)-allyl 2-((((9H-fluoren-9-yl)methoxy)carbonyl)amino)-3-(((R)-2,3-dihydroxypropyl)thio)propanoate): Fmoc-Cys-OAll, 11 (500 mg, 1.3 mmol) was dissolved in dry DCM (15 mL) and zinc powder (600 mg, 9.13 mmol) was added. To this mixture was added freshly prepared mixture of methanol: HCl: conc.H2SO4 (100:7:1, 2 mL)) and stirred vigorously for 20 min. Then (R)-(+)-glycidol, 12 (0.1 mL, 1.40 mmol) was added and the reaction mixture was heated at 40° C. for 3 hrs. 5% Aqueous potassium hydrogen sulfate solution (50 mL) was added, stirred at room temperature for 10 min and extracted with DCM (25 mL×2). The organic phase was dried over anhydrous sodium sulfate and concentrated. The crude was purified by silica gel column chromatography using 3% DCM in methanol as eluent. The product, 13 was obtained as colorless foamy solid (230 mg, 38%). ES-MS (m/z) calculated for C₂₄H₂₇NO₆S: 457.16. Found 458.3 (M+H), 480.3 (M+Na). ¹H NMR (400 MHz, CDCl3): δ 7.75-7.80 (d, 2H), 7.58-7.65 (d, 2H), 7.38-7.45 (m, 2H), 7.30-7.38 (m, 2H), 5.80-6.00 (m, 1H), 5.25-5.40 (m, 2H), 4.60-4.75 (m, 3H), 4.40-4.45 (m, 2H), 4.20-4.22 (m, 1H), 3.80 (m, 1H), 3.65-3.75 (m, 1H), 2.85-3.10 (m, 2H), 2.60-2.85 (m, 2H).

Preparation of compound 15, (2R)-3-((2-((((9H-fluoren-9-yl)methoxy)carbonyl)amino)-3-(allyloxy)-3-oxopropyl)thio)propane-1,2-diyl bis(13-methyltetradecanoate): To a solution of 13-methyl tetradecanoic acid, 8 (3.5 g, 15 mmol) in DCM (80 mL) was added DMAP (225 mg, 1.84 mmol) and cysteine compound, 13 (2.1 g, 5.0 mmol). DCC (3.78 g, 18.5 mmol) was added at 0° C. and stirred at room temperature overnight. The reaction was quenched with acetic acid (1 mL) and concentrated, co-evaporated with toluene (10 mL×3). The crude was then stirred with diethyl ether to give product, 15 as white solid which was collected by filtration (4.1 g, 98%). ES-MS (m/z) calculated for C₅₄H₈₃NO₈S: 905.58. Found 906.7 (M+H), 907.7 (M+2H). ¹H NMR (400 MHz, CDCl3): δ 7.75-7.80 (d, 2H), 7.60-7.68 (d, 2H), 7.38-7.45 (m, 2H), 7.30-7.38 (m, 2H), 5.88-6.00 (m, 1H), 5.70 (d, 1H), 5.38-5.42 (m, 1H), 5.80-5.52 (m, 1h), 5.12-5.20 (brs, 1H), 4.62-4.72 (m, 3H), 4.38-4.46 (m, 2H), 4.32-4.37 (m, 1H), 4.20-4.28 (m, 1H), 4.15-4.20 (m, 1H), 3.0-3.18 (m, 2H), 2.75-2.80 (m, 2H), 2.28-2.38 (m, 4H), 1.58-1.68 (m, 6H), 1.48-1.56 (m, 2H), 1.20-1.38 (s, 44H), 1.10-1.20 (m, 5H), 0.88 (s, 6H), 0.98 (s, 6H).

Preparation of compound 18, (R)-3-(((R)-3-(allyloxy)-2-(13-methyltetradecanamido)-3-oxopropyl)thio)propane-1,2-diyl bis(13-methyltetradecanoate): To a solution of dilipocystein, 15 (906 mg, 1.0 mmol) in DMF (20 mL) peiperidine (4 mL) was added and stirred for 1 hour at room temperature. The solvent was removed under high vacuum, co-evaporated with toluene 3 times and dried under high vacuum. The amine, 16 thus obtained was dissolved in DCM (40 mL) and the lipid acid, 8 (330 mg, 1.37 mmol) was added followed by HOBt (218 mg, 1.62 mmol) and DCC (333 mg, 1.62 mmol). The reaction was stirred at room temperature overnight and filtered off the urea formed. The filtrate was concentrated and purified by silica gel column chromatography using 5-10% ethyl acetate and hexanes. The product, 18 was obtained as a white solid (750 mg, 83%). ES-MS (m/z) calculated for C₅₄H₁₀₁NO₇S: 907.73. Found 908.7 (M+H), 909.7 (M+2H). ¹H NMR (400 MHz, CDCl3): δ 6.35 (d, 2H, J=10), 5.88-6.00 (m, 1H), 5.38 (m, 1H), 5.35 (m, 1H), 5.30 (m, 1H), 5.28 (m, 1H), 5.12-5.20 (brs, 1H), 4.84-4.90 (m, 1H), 4.65-4.68 (m, 2H), 4.32-4.38 (m, 1H), 3.12-4.18 (m, 1H), 3.0-3.18 (m, 2H), 2.75-2.80 (m, 2H), 2.22-2.38 (m, 6H), 1.58-1.68 (m, 6H), 1.20-1.38 (m, 54H), 0.88 (s, 12H), 0.98 (s, 6H).

Preparation of compound 19, (R)-3-(((R)-2,3-bis((13-methyltetradecanoyl)oxy)propyl)thio)-2-(13-methyltetradecanamido)propanoic acid: Trilipocystein allyl ester, 18 (750 mg, 0.83 mmol) was dissolved in anhydrous THF (20 mL) and N-methyl aniline (0.9 mL, 8.3 mmol) and tetrakis(triphenyl phosphine) palladium (0) catalyst (95 mg, 0.083 mmol). The reaction mixture was stirred at room temperature under nitrogen atmosphere for 2 hours. The solvents were removed under reduced pressure and purified by silica gel column chromatography using 2-5% DCM in methanol to afford trilipocystein acid, 19 (560 mg, 78%). ES-MS (m/z) calculated for C₅₁H₉₇NO₇S: 867.70. Found 869.0 (M+H), 870.0 (M+2H).

Preparation of compound 22, Fmoc-Gln(Trt)-Ala-OBzl ester: Fmoc-Gln(Trt)-OH, 20 (10 g, 16.37 mmol) and alaninebenzyl ester tosylate salt, 21 (8.05 g, 22.92 mmol) were taken in DCM (100 mL) and added triethyl amine (3.19 mL, 22.92 mmol). The reaction mixture was stirred for 5 min and were added HOBt (2.43 g, 18.01 mmol) and DCC (3.71 g, 18.01 mmol). The reaction mixture was stirred over night and diluted with DCM and filtered. The filtrate was concentrated and purified by silica gel column chromatography using 50% ethyl acetate and hexane. The product was obtained as white solid, 10 g, 79%. ES-MS (m/z) calculated for C₄₉H₄₅N₃O₆: 771.33. Found 772.5 (M+H), 793.2 (M+Na).

Preparation of compound 23, Fmoc-Gln(Trt)-Ala-OH: Compound 22 (10 g, 12.95 mmol) was dissolved in the mixture of methanol and ethyl acetate (1:1, 300 mL) and stirred with 10% Pd-C catalyst under hydrogen balloon atmosphere for 30 min. The reaction mixture was filtered over a celite bed and the bed was washed with methanol (200 mL). The combined filtrate and washings were concentrated and re-crystallized from ethyl acetate and methanol. The product was obtained as white solid collected by filtration. Yield: 8.1 g, 91%. ES-MS (m/z) calculated for C₄₂H₃₉N₃O₆: 681.28. Found 682.4 (M+H). ¹H NMR (400 MHz, CDCl3): δ 7.35-7.75 (3m, 6H), 7.20-7.35 (m, 16H), 7.15 (d, 1H, J=10), 5.95 (d, 1H, J=10), 4.20-4.48 (m, 5H), 2.38-2.42 (m, 2H), 2.18 (m, 1H), 1.80 (m, 1H), 1. 38 (d, 3H, J=8).

Preparation of compound 25, Fmoc-Gln(Trt)-Ala-Lys(Boc)-OtBu ester: This compound was prepared using the same procedure described for compound 22 from compound 23 (6.76 g, 9.92 mmol) and compound 24 (3.0 g, 9.92 mmol). The crude was purified over silica gel column using 10-25% ethyl acetate and hexane to afford pure product, 25. Yield: 6.7 g, 70%). ES-MS (m/z) calculated for C₅₇H₆₇N₅O₉: 965.49. Found 966.5 (M+H). ¹H NMR (400 MHz, CDCl3): δ 7.35-7.75 (3m, 6H), 7.20-7.35 (m, 16H), 7.15 (m, 1H), 5.95 (d, 1H, J=10), 4.8 (m, 1H), 4.20-4.48 (m, 5H), 3.33-3.35 (m, 1H), 3.0 (m, 3H), 2.50 (m, 2H), 2.05-2.10 (m, 3H), 1.50-1.70 (m, 4H), 1.40-1.50 (2s, 18H), 1.20-1.30 (m, 3H).

Preparation of compound 27, Fmoc-Ser(tBu)-Gln(Trt)-Ala-Lys(Boc)-OtBu ester: Compound 25 (5.5 g, 5.69 mmol) was stirred with 20% morpholine in DMF (60 mL) at room temperature for 1 hour and removed solvents under reduced pressure. It was then co-evaporated with toluene (×3) and dried under high vacuum for 2 hours. The crude free amine was dissolved in DCM (20 mL) and Fmoc-Ser(tBu)-OH, 26 (2.26 g, 5.9 mmol) was added followed by HOBt (0.87 g, 6.4 mmol) and DCC (1.55 g, 7.53 mmol). The reaction mixture was stirred overnight at room temperature. The urea was removed by filtration and the filtrate was concentrated and triturated with ether (×2). The ether layer was decanted and solid was dissolved in DCM and hexane was added until the solution becomes cloudy and left in the refrigerator overnight. The pure product was precipitated out which was collected by filtration. Yield: 4.2 g, 66%. ES-MS (m/z) calculated for C₆₄H₈₀N₆O₁₁: 1108.59. Found 554.8 (M+H)/2. ¹H NMR (400 MHz, cdcl₃) δ 7.85 (s, 1H), 7.77 (d, J=7.6 Hz, 2H), 7.59 (d, J=7.3 Hz, 2H), 7.41 (t, J=7.6 Hz, 2H), 7.27 (m, 18H), 7.03 (m, 2H), 6.94 (m, 1H), 6.81 (m, 1H), 5.74 (s, 1H), 5.01 (m, 1H), 4.81 (m, 1H), 4.50-4.07 (m, 8H), 3.83-3.65 (m, 2H), 3.46 (s, 3H), 3.00 (d, J=31.3 Hz, 3H), 2.54 (d, J=27.9 Hz, 3H), 2.08 (s, 3H), 1.91 (d, J=20.7 Hz, 2H), 1.72 (d, J=14.0 Hz, 3H), 1.58 (d, J=41.5 Hz, 3H), 1.49-1.37 (m, 12H), 1.37-1.20 (m, 9H), 1.15 (d, J=19.9 Hz, 9H), 0.95-0.80 (m, 3H).

Preparation of compound 29, Fmoc-Asn(Trt)-Ser(tBu)-Gln(Trt)-Ala-Lys(Boc)-OtBu ester: Compound 27 (500 mg, 0.45 mmol) was stirred with 10% piperidine in DMF (10 mL) at room temperature for 1 hour and removed solvents under reduced pressure. It was then co-evaporated with toluene (×3) and dried under high vacuum for 2 hours. The crude free amine was dissolved in DCM (20 mL) and Fmoc-Asn(Trt)-OH, 28 (281 mg, 5.9 mmol) was added followed by HOBt (69 mg, 0.51 mmol) and DCC (123 mg, 0.60 mmol). The reaction mixture was stirred overnight at room temperature. The urea was removed by filtration and the filtrate was concentrated and triturated with ether (×2). The ether layer was decanted and solid was dissolved in DCM and hexane was added until the solution becomes cloudy and left in the refrigerator overnight. The pure product, 29 was precipitated out which was collected by filtration. Yield: 450 mg, 68%. ES-MS (m/z) calculated for C₈₇H₁₀₀N₈O₁₃: 1464.74. Found 732.87 (M+H)/2. ¹H NMR (400 MHz, CDCl3) δ 7.75 (d, J=10, 2H), 7.55 (dd, J=7.6 Hz, 2H), 7.45 (d, J=7.3 Hz, 2H), 7.10-7.30 (m, 38H), 6.95 (m, 1H), 6.85 (m, 1H), 4.10-4.50 (m, 9H), 3.70 (m, 1H), 3.0 (m, 3H), 2.38 (m, 2H), 1.82 (m, 3H), 1.58 (m, 1H), 1.44 (s, 9H), 1.43 (s, 9H), 1.38 (d, J=4, 3H), 1.08 (s, 9H).

Preparation of compound 30, Tilipocysteinyl-Asn(Trt)-Ser(tBu)-Gln(Trt)-Ala-Lys(Boc)-OtBu: Compound 29 (870 mg, 0.59 mmol) was stirred with 10% piperidine in DMF (10 mL) at room temperature for 1 hour and removed solvents under reduced pressure. It was then co-evaporated with toluene (×3) and dried under high vacuum for 2 hours. The crude free amine was dissolved in DCM (40 mL) and lipocystein-acid, 19 (496 mg, 0.57 mmol) was added followed by HOBt (100 mg, 0.74 mmol) and DCC (153 mg, 0.74 mmol). The reaction mixture was stirred overnight at room temperature. The urea was removed by filtration and the filtrate was concentrated and triturated with ether (×2). The ether layer was decanted and solid was dissolved in DCM and hexane was added until the solution becomes cloudy and left in the refrigerator overnight. The product was further purified by silica gel column chromatography using 5-10% methanol in DCM. Yield: 550 mg, 44%. ES-MS (m/z) calculated for C₁₂₃H₁₈₅N₉O₁₇S: 2092.36. Found 2093.4 (MALDI).

EXAMPLE 1—Preparation of compound 31, Trilipocysteinyl-Asn-Ser-Gln-Ala-Lys-OH ((iC15)3CNSQAK): Compound 30 (100 mg, 0.048 mmol) was added to the freshly prepared Reagent B (TFA:Phenol:Triisopropyl silane:water, 8.8:0.5:0.2:0.2) cocktail (10 mL) and stirred for 1 hour at room temperature and diluted with chloroform (10 mL). The mixture was concentrated under reduced pressure keeping the water bath temperature below 20° C. to remove up to 80% of the solvents. To this reduced volume was added cold diethyl ether (˜50 mL) and kept at −20° C. for one hour. The mixture was filtered through a sintered funnel and collected a while jelly like substance which was dissolved in 10% aqueous acetic acid (20 mL) and extracted with chloroform (20 mL×2). The aqueous layer was collected and lyophilized. The white fluffy solid thus obtained was dissolved in chloroform:methanol (2:1) (10 mL) and the undissolved portion was discarded. The solution was adsorbed on silica gel and purified by silica gel column chromatography using CHCl3:MeOH:H2O:HOAc (7.5:2:0.25:0.25) system. The compound containing fractions were collected and concentrated. The product was obtained oily film which was then dissolved in tert-butanol and water mixture (6:4) and lyophilized. The product was obtained as a white fluffy solid, 60 mg. ES-MS (m/z) calculated for C72H133N9O15S: 1395.96. Found 1396.4, 1397.4, 1398.5, 1399.3 (MALDI).

Preparation of compound 34, Fmoc-Met-Leu-OBzl ester: L-leucine benzyl ester tosylate salt, 33 (8.8 g, 54.9 mmol) was taken in dry DCM and triethyl amine (3.06 mL, 54.9 mmol) was added and stirred at room temperature for 30 min. To this clear solution was added L-Fmoc-Met-OH (4.8 g, 32.3 mmol) followed by HOBt (2.26 g, 42.0 mmol) and DCC (3.46 g, 42.0 mmol). The reaction was stirred overnight. The reaction was filtered to remove the urea and the filtrate was concentrated and stirred with diethyl ether for 12 hours. The product, 34 was collected by filtration which was pure enough to continue to the next step. Yield: 6.9 g, 93%. ES-MS (m/z) calculated for C₃₃H₃₈N₂O₅S: 574.25. Found 575.3 (M+H). ¹H NMR (400 MHz, CDCl3) δ 7.75 (d, J=8 Hz, 2H), 7.55 (d, J=7.6 Hz, 2H), 7.10-7.40 (m, 9H), 6.65 (m, 1H), 5.65 (m, 1H), 5.18 (dd, 2H), 4.62-4.72 (m, 1H), 4.38-4.50 (m, 3H), 4.20-4.22 (t, 1H), 2.60 (m, 2H), 2.15 (s, 3H), 2.0-2.10 (m, 2H), 1.55-1.70 (m, 3H), 0.95 (d, J=6 Hz, 6H).

Preparation of compound 36, Fmoc-Met-Leu-Phe-OBzl: The benzyl ester, 34 (18.0 g, 31.3 mmol) was dissolved in the mixture of methanol and ethyl acetate (1:1, 100 mL) and 10% Pd-C(3.0 g) was added slowly. The mixture was degassed 3 times with hydrogen flush and stirred under positive pressure of hydrogen for 3 hours. The reaction completion was monitored by TLC and removed from the hydrogen pressure. The mixture was carefully filtered through celite pad and washed the celide bed 3 times with methanol. The combined filtrate and washings were concentrated to dryness and dried under vacuum for 2 hours to give Fmoc-Met-Leu-OH as a white solid. L-Phenyl alanine benzyl ester tosylate salt (10.6 g, 24.97 mmol) was dissolved in DCM (50 mL) and stirred with triethyl amine (3.48 mL, 24.97 mmol) for 30 min. Then Fmoc-Met-Leu-OH (11.0 g, 22.7 mmol) was added followed by HOBt (3.98 g, 29.5 mmol) and DCC (6.08 g, 29.5 mmol) and the reaction mixture was allowed to stir at room temperature overnight. The white solid, urea was removed by filtration and the filtrate was concentrated and stirred with ether (100 mL) to give product, 36 as a white solid. Yield: 12 g, 53% over two steps. ES-MS (m/z) calculated for C₄₂H₄₇N₃O₆S: 721.32. Found 722.3 (M+H). ¹H NMR (400 MHz, CDCl3) δ ¹H NMR (400 MHz, DMSO-d6) δ 8.40 (d, J=7.3 Hz, 1H), 7.86 (dd, J=13.9, 8.2 Hz, 2H), 7.69 (t, J=7.0 Hz, 2H), 7.57-7.46 (m, 2H), 7.40 (t, J=7.5 Hz, 2H), 7.35-7.25 (m, 11H), 5.56 (d, J=7.7 Hz, 1H), 5.02 (m, 2H), 4.50 (m, 1H), 4.07-4.26 (m, 6H), 3.72 (m, 4H), 2.33 (m, 2H), 2.00 (s, 3H), 1.34 (m, 2H), 0.77 (dd, J=11.2, 6.6 Hz, 6H).

Preparation of compound 37, Formyl Met-Leu-Phe-OBzl: Compound 36 (11.5 g, 15.9 mmol) was stirred with 20% piperidine in DMF (100 mL) for 1 hour and removed solvents under high vacuum. Codistilled with toluene (20 mL×3) and the residue was dried under high vacuum for 2 hours. The crude free amine thus obtained was dissolved in DCM (40 mL) and was added slowly to a chilled mixture of DCC (4.64 g, 22.6 mmol) in DCM (40 mL) and formic acid (0.850 mL, 22.5 mmol) over 30 min period. The reaction mixture was stirred for 36 hours at room temperature. The solids were removed by filtration. The filtrate was first triturated with ether for 12 hours and then purified by silica gel column chromatography using 2-5% methanol in DCM to give compound 37 as a white solid (5 g, 59%). ES-MS (m/z) calculated for C₂₈H₃₇N₃O₅S: 527.25. Found 528.25 (M+H). ¹H NMR (400 MHz, CDCl3) δ ¹H NMR (400 MHz, DMSO-d6) δ 8.19 (s, 1H), 7.34-7.60 (dd, J=13.9, 8.2 Hz, 2H), 7.25-7.35 (m, 2H), 7.19-7.22 (m, 3H), 6.95-7.05 (m, 3H), 6.85-6.89 (m, 2H), 5.15 (dd, J=10 Hz, 7.7 Hz, 2H), 4.85 (m, 1H), 4.75 (m, 1H), 4.50 (m, 1H), 3.45 (m, 1H), 3.15 (m, 2H), 2.5 (m, 2H), 2.04 (s, 3H), 1.74 (m, 2H), 1.55 (m, 1H), 0.82 (dd, J=11.2, 6.6 Hz, 6H).

Preparation of compound 38, Formyl Met-Leu-Phe-OH: Compound 37 (500 mg, 0.95 mmol) was dissolved in the mixture of MeOH:THF:HOAC (50:10:1) and 10% Pd-C (170 mg) was added. The reaction mixture was degassed three times with H2 flush and hydrogenated at 50 psi using a Parr-shaker for 18 hours. The reaction mixture was filtered through a celite bed and the bed was washed with methanol three times. The combined washings and filtrate were concentrated to dryness. The residue was stirred with ether and the resulting white solid was collected as compound 38 (220 mg, 53%). ES-MS (m/z) calculated for C₂₁H₃₁N₃O₅S: 437.20. Found 436.2 (M−H, negative mode). ¹H NMR (400 MHz, DMSO-d6) δ 8.39 (m, 1H), 8.18 (d, J=4 Hz, 1H), 7.98 (s, 1H), 7. 50 (brs, 1H), 7.08-7.20 (m, 5H), 4.40 (m, 1H), 4.20 (m, 2H), 3.02-3.08 (m, 2H), 2.85-2.96 (m, 1H), 2.40 (m, 2H), 2.00 (s, 3H), 1.75 (m, 1H), 1.58 (m, 1H), 1.40 (m, 2H), 0.75 (d, J=6 Hz, 3H), 0.85 (d, J=6 Hz, 3H).

Preparation of compound 41, benzyl (2-aminoethyl)carbamate: Benzyl chloroformate (2.85 mL, 20 mmol) taken in DCM (150 mL) was added slowly to a chilled solution of 1,2-diaminoethane (13.5 mL, 20 mmol) in DCM (100 mL) over a period of 1 hour at 0° C. The reaction was allowed to stir at room temperature for 24 hours and washed with water and brine. The organic layer was dried over anhydrous sodium sulfate and concentrated. The residue was purified by silica gel column chromatography using 10-20% methanol in DCM with a trace of triethyl amine in the solvent system. The product, 41 was obtained a pale yellow solid. The product was identical to the product reported in New J. Chem., 2013, 37, 1895-1905.

Preparation of compound 42, Fmoc-Lys(Boc)-NH—CH2-CH2-NH-Cbz: Fmoc-Lys(Boc)-OH, 39 (21.22 g, 45.53 mmol), compound 41 (8.0 g, 41.19 mmol) were taken in dry DCM (600 mL) and triethyl amine (5.74 mL, 41.19 mmol), HOBt (7.79 g, 57.66 mmol) and DCC (11.89 g, 57.66 mmol) were order in the same order. The reaction mixture was stirred at room temperature overnight and the urea was removed by filtration. The filtrate was concentrated to give a sticky white solid which was stirred with ether overnight and filtered. The pure product was obtained as a white solid which was dried under high vacuum and used in the next step without further purification. Yield: 26 g, 98%. ES-MS (m/z) calculated for C₃₆H₄₄N₄O₇: 644.32. Found 645.3 (M+H). ¹H NMR (400 MHz, CDCl3+CD3OD) δ 7.78 (d, J=4 Hz, 2H), 7.58 (d, J=2 Hz, 2H), 7. 40 (t, J=4 Hz, 2H), 7.30 (m, 7H), 6.70 (m, 1H), 5.4-5.60 (m, 2H), 5.15 (m, 2H), 4.68 (m, 1H), 4.40 (m, 2H), 4.20 (m, 1H), 4.10 (m, 1H), 3.40-3.50 (m, 4H), 3.02-3.08 (m, 2H), 1.60-1.80 (m, 6H), 1.45 (s, 9H).

Preparation of compound 44, Fmoc-Lys(Boc)-Lyc(Boc)-NH—CH2-CH2-NH-Cbz: Following the same procedure as described for compound 37, starting from compound 42 (10 g, 15.5 mmol) and Fmoc-Lys(Boc)-OH (6.65 g, 14.20 mmol), HOBt (2.49 g, 18.46 mmol), DCC (3.80 g, 18.46 mmol), compound 44 was made. Yield: 13 g, 96%. ES-MS (m/z) calculated for C₄₇H₆₄N₆O₁₀: 872.47. Found 873.4 (M+H).

Preparation of compound 46, Fmoc-Lys(Boc)-Lys(Boc)-Lyc(Boc)-NH—CH2-CH2-Cbz: Following the same procedure as described for compound 37, starting from compound 44 (12.0 g, 13.7 mmol) and Fmoc-Lys(Boc)-OH (5.76 g, 12.29 mmol), HOBt (2.16 g, 15.98 mmol), DCC (3.30 g, 15.98 mmol), compound 46 was made. Yield: 14.5 g, 96%. ES-MS (m/z) calculated for C₅₉H₈₅N₇O₁₃:1099.62. Found 1100.6 (M+H).

Preparation of compound 48, Fmoc-Ser(tBu)-Lys(Boc)-Lys(Boc)-Lys(Boc)-NH—CH2-CH2-Cbz: Following the same procedure as described for compound 37, starting from compound 46 (14.0 g, 12.7 mmol) and Fmoc-Ser(tBu)-OH (4.36 g, 11.37 mmol), HOBt (2.0 g, 14.79 mmol), DCC (3.05 g, 14.79 mmol), compound 48 was made. Yield: 13.0 g, 82%. ES-MS (m/z) calculated for C₆₅H₉₇N₉O₁₅:1243.71. Found 1244.7 (M+H).

Preparation of compound 49, TrilipoCys-Ser(tBu)-Lys(Boc)-Lys(Boc)-Lys(Boc)-NH—CH2-CH2-NH-CBz: Following the same procedure as described for compound 37, starting from compound 48 (750 mg, 0.60 mmol) and Trilipo Cys-OH (500 mg, 0.58 mmol), HOBt (101 mg, 0.75 mmol), DCC (154 mg, 0.75 mmol), compound 49 was made. Yield: 850 mg, 76%. ES-MS (m/z) calculated for C₁₀₁H₁₈₂N₁₀O₁₉S:1871.33. Found 936.3 (M+H)/2.

Preparation of compound 50, TrilipoCys-Ser(tBu)-Lys(Boc)-Lys(Boc)-Lys(Boc)-NH—CH2-CH2-NH2: Compound 49 (850 mg, 0.45 mmol) was dissolved in the mixture of THF (15 mL), MeOH (25 mL) and acetic acid (1 mL). The reaction mixture was degassed three times and hydrogenated in Parr shaker for 24 hours. The reaction mixture was filtered through a celite bed and washed with methanol (250 mL). The combined washings and filtrate was concentrated and purified by silica gel column chromatography using 5-15% methanol in DCM with 0.1% triethylamine. The free amine product, 50 was obtained as a white solid (350 mg, 45%). ES-MS (m/z) calculated for C₉₃H₁₇₆N₁₀O₁₇S: 1737.29. Found 1738.4 (M+H).

Preparation of compound 51, TrilipoCys-Ser(tBu)-Lys(Boc)-Lys(Boc)-Lys(Boc)-NH—CH2-CH2-NH-F-L-Mf: Amine, 49 (300 mg, 0.17 mmol) was dissolved in dry DCM (30 mL) and dry DMF (1 mL) to which acid, 38 (90 mg, 0.21 mmol) was added followed by HOBt (30 mg, 0.22 mmol) and DCC (46 mg, 0.22 mmol). The reaction mixture was stirred at room temperature overnight. The solvents were removed under reduced pressure, co-distilled with toluene to remove DMF and purified by column chromatography using 2-10% methanol in DCM as eluent. The product was obtained as colorless solid (200 mg, 54%). MALDI-MS (m/z) calculated for C₁₁₄H₂₀₅N₁₃O₂₁S₂: 2156.48. Found 2157.5 (M+H), 2158.5 (M+2H); ES-MS (m/z): 1101.9 (M+2Na)/2.

EXAMPLE 2-Preparation of compound 52, TrilipoCys-Ser-Lys-Lys-Lys-NH—CH2-CH2-NH-F-L-Mf: To compound 51 (140 mg, 0.065 mmol) was added 15 mL of Reagent B cocktail and stirred for 3 hours at RT. The reaction was monitored by TLC and upon completion of the starting material, it was diluted with chloroform and solvents were removed under reduced pressure. To this residue was added cold ether (25 mL) and kept at −20° C. overnight. To this chloroform (30 mL) was added and extracted with 10% acetic acid in water. The organic layer was concentrated and purified on silica gel column chromatography using CHCl3:MeOH:Et3N (5-20% methanol in DCM with 0.5% Et3N). The compound containing fractions were collected and concentrated to dryness. Then dissolved in 0.1% HCl in water and lyophilized. The product was obtained as fluffy white solid, 40 mg, 32%. MALDI-MS (m/z) calculated for C₉₅H₁₇₃N₁₃O₁₅S₂(1800.26). Found 1800 (M+), 1801 (M+H), 1802 (M+2H).

Compound 60 is known (J. Med. Chem., 1983, 26, 1729-1732), and was prepared by following the literature method.

Preparation of compound 61, (S)-benzyl 4-((S)-2-((R)-2-(((2S,3R,4R,5S,6R)-3-acetamido-2-(benzyloxy)-5-hydroxy-6-(hydroxymethyl)tetrahydro-2H-pyran-4-yl)oxy)propanamido)propanamido)-5-amino-5-oxopentanoate: Compound 60 (80 mg, 0.105 mmol) was dissolved in the mixture of TFA and water (9:1, 10 mL) and stirred for 1 hour and removed solvents under reduced pressure. The residue was purified by column chromatography using 5-15% methanol in DCM. Yield: 53 mg, 66%. ES-MS (m/z) calculated C₃₃H₄₄N₄O₁₁ (672.30). Found 673.3 (M+H), 695.2 (M+Na).

Preparation of compound 62, ((2R,3S,4R,5R,6S)-5-acetamido-4-(((R)-1-(((S)-1-(((S)-1-amino-5-(benzyloxy)-1,5-dioxopentan-2-yl)amino)-1-oxopropan-2-yl)amino)-1-oxopropan-2-yl)oxy)-6-(benzyloxy)-3-hydroxytetrahydro-2H-pyran-2-yl)methyl 13-methyltetradecanoate: The lipid acid, 8 (74 mg, 0.33 mmol) was dissolved in DCM (2 mL) and oxalyl chloride (56.6 uL) and 1 drop of DMF was added. The reaction mixture was stirred for 1 hour and solvent was removed under reduced pressure. To the DCM (5 mL) solution of diol, 62 (212 mg, 0.32 mmol) was added DIPEA (61 uL, 0.35 mmol). To this mixture was added the freshly prepared lipid acid chloride dissolved in DCM (1 mL) drop wise through a dropping funnel at room temperature. The resultant mixture was allowed to stir overnight and concentrated. The residue was purified by silica gel column chromatography using 1-5% methanol in DCM. The product was obtained as a foamy solid. Yield: 104 mg, 37%. ES-MS (m/z) calculated C₄₈H₇₂N₄O₁₂ (896.51). Found 897 (M+H). ¹H NMR (400 MHz, CD3OD) δ 7.42-7.18 (m, 10H), 5.10 (d, J=4 Hz, 1H), 4.92-4.78 (m, 2H), 4.69 (d, J=11.8 Hz, 2H), 4.54-4.40 (m, 2H), 4.33 (m, 3H), 4.25-4.11 (m, 2H), 3.95 (m, 1H), 3.87-3.79 (m, 1H), 3.68-3.57 (m, 2H), 2.51-2.38 (m, 1H), 2.35 (t, J=7.4 Hz, 2H), 2.28-2.11 (m, 1H), 1.95-1.80 (m, 2H), 1.61 (dd, J=14.4, 7.0 Hz, 1H), 1.55-1.39 (m, 4H), 1.41-1.18 (m, 17H), 1.15 (d, J=6.9 Hz, 6H), 0.86 (d, J=6.6 Hz, 6H).

EXAMPLE 3: Preparation of compound 63, (S)-4-((S)-2-((R)-2-(((2R,3R,4R,5S,6R)-3-acetamido-2,5-dihydroxy-6-(((13-methyltetradecanoyl)oxy)methyl)tetrahydro-2H-pyran-4-yl)oxy)propanamido)propanamido)-5-amino-5-oxopentanoic acid: Compound 62 (100 mg, 0.11 mmol) was dissolved in the mixture of THF (20 mL) and acetic acid (2 mL). 10% Pd-C was added after degassing for few minutes and stirred under H2 gas balloon pressure overnight. The catalyst was removed by filtration over celite and the celite was washed with THF (50 mL). The combined filtrate and washings were concentrated under reduced pressure and the residue was purified by silica gel column chromatography using DCM:MeOH:H2O:HOAc (9:1.5:0.15:0.1). The product thus obtained was dissolved in tert.butanol and lyophilized to give the title product as a white solid. Yield: 46 mg, 57%. ES-MS (m/z) calculated C₃₄H₆₀N₄O₁₂ (716.42). Found 717.4 (M+H), 699 (M−H2O+H). ¹H NMR (400 MHz, CD3OD+CDCl3) δ 5.18 (d, J=4 Hz, 1H), 4.92-4.78 (m, 2H), 4.54-4.40 (m, 2H), 4.33 (m, 3H), 4.25-4.11 (m, 2H), 3.95 (m, 1H), 3.87-3.79 (m, 1H), 3.68-3.57 (m, 2H), 2.40 (m, 2H), 2.20 (m, 1H), 2.00 (s, 3H), 1.61 (dd, J=14.4, 7.0 Hz, 2H), 1.55-1.39 (m, 4H), 1.41-1.18 (m, 18H), 1.15 (d, J=6.9 Hz, 2H), 0.86 (d, J=6.6 Hz, 6H).

Compounds 65, 66 and 67 are known compounds and were prepared according to the literature reported methods.

Preparation of compound 69, (2R,4aR,6S,7R,8R,8a5)-6-(benzyloxy)-2-phenyl-7-4(2,2,2-trichloroethoxy)carbonyl)amino)hexahydropyrano[3,2-d][1,3]dioxin-8-yl palmitate: Compound 67 (53 g, 99.47 mmol) and palmitic acid, 68, (30.6 g, 119.37 mmol) were dissolved in anhydrous DCM (700 mL) and to this uniform solution was added DCC (28.73 g, 139.26 mmol) and DMAP (12.15 g, 99.47 mmol) were added and stirred at room temperature overnight. Diluted with ethyl acetate and filtered off the solids. The filtrate was concentrated and purified by silica gel column chromatography using 10-15% ethyl acetate in hexane. Yield: 45 g, 59%. ES-MS (m/z) calculated C₃₉H₅₄C₁₃NO₈ (769.29). Found 770.4 (M+H). ¹H NMR (400 MHz, CDCl3) δ 7.30-7.50 (m, 10H), 5.55 (d, J=4 Hz, 1H), 5.40-5.48 (m, 1H), 5.0 (d, J=4 Hz, 1H), 4.55-4.75 (m, 6H), 3.95 (m, 1H), 3.75-3.85 (m, 4H), 2.35 (m, 2H), 1.60 (m, 2H), 1.30 (s, 24H), 0.90 (t, J=10 Hz, 8 Hz, 3H).

Preparation of compound 70, (2S,3R,4R,5S,6R)-2-(benzyloxy)-5-hydroxy-6-(hydroxymethyl)-3-4(2,2,2-trichloroethoxy)carbonyl)amino)tetrahydro-2H-pyran-4-yl palmitate: Compound 69 (45 g, 58.35 mmol) was taken 80% acetic acid in water and heated to 75-80° C. for 6 hours. The solution was cooled and evaporated to dryness. The crude is then column purified on silica gel column using 0-25% ethyl acetate in hexane. Yield: 20 g, 50%. ES-MS (m/z) calculated C₃₂H₅₀Cl₃NO₈ (681.26). Found 682.3 (M+H). ¹H NMR (400 MHz, CDCl3) δ 7.30-7.50 (m, 5H), 5.55 (d, J=4 Hz, 1H), 5.40-5.48 (m, 1H), 5.0 (d, J=4 Hz, 1H), 4.55-4.75 (m, 4H), 3.95 (m, 1H), 3.75-3.85 (m, 4H), 2.60 (brs, 1H), 2.35 (m, 2H), 1.90 (brs, 1H), 1.60 (m, 2H), 1.30 (s, 24H), 0.90 (t, J=10 Hz, 8 Hz, 3H).

Compounds 73-90 were prepared by following the procedures as described in the literature. (U.S. Pat. No. 7,491,707 B1)

Preparation of Compound 90, (R)-(2R,3S,4R,5R,6R)-2-((benzyloxy)methyl)-3-((bis(benzyloxy)phosphoryl)oxy)-6-(2,2,2-trichloro-1-iminoethoxy)-5-(((2,2,2-trichloroethoxy)carbonyl)amino)tetrahydro-2H-pyran-4-yl 3-(tetradecanoyloxy)tetradecanoate: Compound 89 (1.0 g, 0.88 mmol) was dissolved in dry DCM (10 mL) and trichloroacetonitrile (0.5 mL, 2.0 mmol) was added followed by cesium chloride (428 mg, 1.32 mmol). The reaction mixture was stirred at room temperature for 30 min and filtered through a celite pad. The filtrate was concentrated and immediately purified by silica gel column chromatography using 30% ethyl acetate in hexane to afford the title compound as color less oil. Yield: 900 mg, 80%. ES-MS (m/z) calculated C₆₀H₈₅Cl₆N₂O₁₃P (1282.39). Found 1283.4 (M+H). ¹H NMR (400 MHz, CDCl3) δ 8.75 (s, 1H), 7.30 (m, 15H), 6.45 (d. J-3.5 Hz, 1H), 5.43 (dd, J=10.5, 9.5 Hz, 1H), 5.30 (d. J=8.5 Hz, 1H), 4.95 (m, 4H), 4.83 (d. J=12.0 Hz, 1H), 4.80 (m. 1H), 4.53 (d. J=12.0 Hz, 1H), 4.50 (d, J=12.0 Hz, 1H), 4.45 (d. J=12.0, HZ, 1H), 4.15 (ddd, J=9.5, 8.5, 3.5 Hz, 1H), 4.04 (m, 1H), 3.73 (d. J=2.5 Hz, 2H), 3.57 (m, 1H), 3.34 (m, 1H), 3.21 (m, 1H), 2.53 (dd, J=15.5, 6.0 Hz, 1H), 2.37 (dd, J=15.5, 6.0 Hz, 1H), 1.40 (m, 4H), 1.25 (brs, 36H), 0.88 (t, J=6.5 Hz, 6H).

Preparation of Compound 91, (2R,3R,4R,5S,6R)-2-(benzyloxy)-6-(((2R,3R,4R,5S,6R)-6-(benzyloxymethyl)-5-(bis(benzyloxy)phosphoryloxy)-4-(((R)-3-(tetradecanoyloxy)tetradecanoyloxy)-3-(2,2,2-trichloroethoxy)carbonylamino)tetrahydro-2H-pyran-2-yloxy)methyl)-5-hydroxy-3-((2,2,2-trichloroethoxy)carbonylamino)tetrahydro-2H-pyran-4-yl palmitate: Compound 90 (donor) (415 mg, 0.32 mmol) and Compound 70 (acceptor) (218.6 mg, 0.32 mmol) were dried under high vacuum and dissolved in dry DCM (15 mL) and stirred with 4 A molecular sieve powder for 10 min. Freshly prepared 0.1M TMSOTf solution in DCM (0.16 mL) was added slowly drop wise and then stirred for 30 min at room temperature. The reaction was quenched with triethyl amine and after usual work followed by purification by silica gel column chromatography afforded title compound as a sticky solid. Yield: 550 mg, 94% yield. ES-MS (m/z) calculated C₉₀H₁₃₃Cl₆N₂O₂₀P (1802.73). Found 1803.7 (M+H). ¹H NMR (400 MHz, CDCl3) δ 7.42-7.18 (m, 20H), 6.69
6.51 (m, 2H), 5.39 (m, 2H), 5.30-5.05 (m, 6H), 5.04-4.81 (m, 6H), 4.82-4.59 (m, 5H), 4.56-4.37 (m, 2H), 4.07 (d, J=9.1 Hz, 1H), 4.01-3.55 (m, 11H), 2.93 (brs, 1H), 2.54-2.12 (m, 9H), 1.73-1.38 (m, 9H), 1.38-1.15 (m, 50H), 0.89 (t, J=6.8 Hz, 9H).

Preparation of Compound 93, (2R,3R,4R,5S,6R)-2-(benzyloxy)-6-(((2R,3R,4R,5S,6R)-6-(benzyloxymethyl)-5-(bis(benzyloxy)phosphoryloxy)-3-((R)-3-(13-methyltetradecanoyloxy)tetradecanamido)-4-((R)-3-(tetradecanoyloxy)tetradecanoyloxy) tetrahydro-2H-pyran-2-yloxy)methyl)-5-hydroxy-3-((R)-3-(13-methyltetradecanoyloxy) tetradecanamido)tetrahydro-2H-pyran-4-yl palmitate: Compound 91 (800 mg, 0.44 mmol) was dissolved in acetic acid (35 mL) and zinc powder (8.0 g) was added. The reaction mixture was stirred at room temperature for about 1 hour and when tlc indicated that the starting material was completely consumed, the reaction mixture was filtered through a celite pad. Washed with ethyl acetate (100 mL) and concentrated. Co-distilled with toluene (50 mL×3) until all the acetic acid was removed. The residue was dried under high vacuum briefly and taken in dry DCM (10 mL). To this solution was added dilipid acid, 77 (515 mg, 1.3 mmol), EDC (253 mg, 1.32 mmol) and stirred overnight. The reaction was diluted with DCM (20 mL) and washed with water (10 mL), brine (10 mL), dried over sodium sulfate and concentrated. The residue was purified by silica gel column chromatography to afford the title compound as oily solid. Yield: 350 mg, 34% over two steps. MALDI-MS (m/z) calculated for C₁₄₂H₂₃₉N₂O₂₂P, 2355.74. Found: 2356.6 (M+H), 2357.6 (M+2H), 2358.2 (M+3H). ³¹PNMR (400 MHz, CDCl3): 2.07, 2.15; Some characteristic peaks of ¹H NMR (400 MHz, CDCl3) δ 7.40-7.18 (m, 20H), 6.33 (s, 1H), 5.91 (d, J=9.3 Hz, 1H), 2.52-2.14 (m), 1.71-1.39 (m), 1.26 (brs, 108H), 1.17 (brs, 8H), 0.97-0.75 (m, 27H).

EXAMPLE-4, Preparation of Compound 94, (3R,4R,5S)-2,5-dihydroxy-6-(((2R,3R,4R,5S,6R)-6-(hydroxymethyl)-3-((R)-3-(13-methyltetradecanoyloxy)tetradecanamido)-5-(phosphonooxy)-4-((R)-3-(tetradecanoyloxy)tetradecanoyloxy)tetrahydro-2H-pyran-2-yloxy)methyl)-3-((R)-3-(13-methyltetradecanoyloxy)tetradecanamido)tetrahydro-2H-pyran-4-yl palmitate: Compound 93 (200 mg, 84.8 μmol) was taken in freshly distilled THF (25 mL) and acetic acid (2.5 mL). To this was added, 10% Pd-C (100 mg) and degassed 3 times with hydrogen flush. Then the reaction mixture was stirred under hydrogen balloon pressure overnight and filtered through a celite pad. The celite was washed thoroughly with THF and the combined filtrate and washings were concentrated. The residue was purified by silica gel column chromatography using the solvent system, chloroform:methanol:water:triethylamine (18:1:0.1:0.1 to 5:1:0.1:0.1). The product containing fractions were concentrated and the residue was lyophilized with tert.butanol to afford compound as a white fluffy solid. Yield: 65 mg, 38%. MALDI-MS calculated for C₁₁₄H₂₁₅N₂O₂₂P, 1955.55. Found: 1995.4.

EXAMPLE-5, Preparation of Compound 95, (2R,3R,4R,5S,6R)-2-(((3S,4R,5R)-3,6-dihydroxy-5-((R)-3-(13-methyltetradecanoyloxy)tetradecanamido)-4-(palmitoyloxy)tetrahydro-2H-pyran-2-yl)methoxy)-6-(hydroxymethyl)-3-((R)-3-(13-methyltetradecanoyloxy)tetradecanamido)-5-(phosphonooxy)tetrahydro-2H-pyran-4-yl palmitate: It is prepared exactly following the same process as described for compound 94 starting from the donor, 90A and using the lipid acid, 77. The final compound 95 was isolated as a white fluffy powder. Yield: 30 mg. MALDI-MS calculated for C₁₀₂H₁₉₃N₂O₂₀P, 1797.39. Found: 1798.4 (M+H), 1799.4 (M+2H).

EXAMPLE-6, Preparation of Compound 96, (2R,3R,4R,5S,6R)-2-(((3S,4R,5R)-3,6-dihydroxy-5-((R)-13-methyl-3-(13-methyltetradecanoyloxy)tetradecanamido)-4-(palmitoyloxy)tetrahydro-2H-pyran-2-yl)methoxy)-6-(hydroxymethyl)-3-((R)-13-methyl-3-(13-methyltetradecanoyloxy)tetradecanamido)-5-(phosphonooxy)tetrahydro-2H-pyran-4-yl palmitate: It is prepared exactly following the same process as described for compound 94 starting from the donor, 90A and using the lipid acid, 77. The final compound 95 was isolated as a white fluffy powder. Yield: 18 mg. MALDI-MS calculated for C₁₀₄H₁₉₇N₂O₂₀P, 1825.42. Found: 1826.4 (M+H), 1827.4 (M+2H).

EXAMPLE-7, Preparation of compound 97, (3R)-((2R,3R,4R,5S,6R)-2-(((3S,4R,5R)-3,6-dihydroxy-4-((R)-3-hydroxytetradecanoyloxy)-5-((R)-3-(tetradecanoyloxy)tetradecanamido)tetrahydro-2H-pyran-2-yl)methoxy)-6-(hydroxymethyl)-5-(phosphonooxy)-3-((R)-3-(tetradecanoyloxy)tetradecanamido)tetrahydro-2H-pyran-4-yl) 3-(tetradecanoyloxy)tetradecanoate, was described in Carbohydrate Research 342 (2007) 784-796. MALDI-MS Calculated for C110H207N2O23P: 1955.5. Found: 1955.5 (M+), 1956.5 (M+H).

EXAMPLE-8: Various combinations of compounds of the formula I, II and III were developed and their immune stimulation capacities were measured in vitro and in vivo. Also, the adjuvanticity of the combinations were explored with an experimental antigen, OVA. The robust immune responses of compounds described herein are assessed by their ability to express the cell surface markers and various cytokines when exposed to the mature dendritic cells derived from mice.

Ability of compounds as vaccine adjuvants are assessed through their ability to induce robust immune responses in mice when administered along with a standard experimental antigen, OVA peptide, and compared the balance of Th1 and Th2 responses from their IgG1a and IgG2a responses as well as the cytokine production in mice. It is very common to see the antibody responses to many adjuvants but it is important to see the cellular responses as well which are used as therapeutic vaccines such as cancer vaccines. The compounds exhibited balanced responses and based on the ratio of the compounds used in the adjuvant, it is possible to modulate or control the immune responses of interest.

Ovalbumin (OVA) was purchased from Sigma-Aldrich. Polyphosphazene, Poly[di(sodium carboxylatoethylphenoxy)phosphazene] (PZ) was purchased from Idaho National Laboratory, Idaho Falls, Id., US, Poly I:C (PIC) was purchased from Invivogen. Vaccine diluent was PBS, pH 7.4 (Gibco, Life Technologies).

Nanoparticle Formulation: PLGA NPs encapsulated were prepared by emulsification solvent evaporation method as mentioned previously. (Jahan S T, et al.; Int J Nanomedicine. 2018 11; 13:367-386) Briefly, OVA/PBS solution (10%), Compound 97 in chloroform:methanol (2%) or Compound 63 in chloroform:methanol (2%) were transferred to the PLGA/chloroform solution (25%). The resulting mixtures were then emulsified in 5% of PVA to form a secondary emulsion followed by stirring for 2 hours to evaporate the solvents. The NPs were then collected by centrifugation. At the final step, the NPs were freeze-dried and stored for further use. The amounts of adjuvants were determined by LC-MS/MS using a pre-column guard. Applied Biosystems/MDS Sciex Analyst software (Version 1.6.0) was used for system control and quantification. A sample volume of 5 μL, was injected using the 1200 Agilent autoinjector set to 4° C. and was delivered with an isocratic mobile phase consisting of methanol (0.1% formic acid) at a flow rate of 200 μl/min for a run-time of 2 minutes.

Mouse Immunization: Groups of BALB/c mice were immunized with vaccine formulations wherein OVA (50 μg/dose) is combined with PZ (10 μg/dose), PIC (10 μg/dose), and adjuvant alone or in combination (20 μg/dose). In Trial 1, mice (n=8 per group) were immunized subcutaneously with 100 μl total volume with OVA alone, OVA+PZ/PI/52, OVA+52, or saline. In Trial 2, mice (n=8 per group) were immunized subcutaneously with 200 μl total volume with OVA alone, OVA+52, OVA+PZ+PIC, OVA+52+PZ+PI, OVA+(63+97) nanoparticles, or OVA+(52+63+97)nanoparticles, where the number refers to the compound number. The volume was doubled in Trial 2 because the (63+97)Np's were viscous and required more buffer to be easily injected. Mice received a booster immunization on Day 21. Blood was obtained on day 21, day 35 for both trials and 48 for trial 1 only. Mice were euthanized with isofluorane on Day 35 in Trial 2 and Day 48 in Trial 1. Spleens were isolated on day of euthanization.

Splenocyte isolation and ex vivo restimulation: Spleens were excised and incubated in Minimal Essential Media (MEM) (Sigma Life Science). They were placed on a petri dish, minced into smaller pieces and pushed through a 0.2 μm cell strainer. Media was used to rinse the strainer and petri dish and collected. Splenocytes were centrifuged at 350×g for 10 minutes at 10° C. Supernatant was discarded and cells were incubated in Gey's solution [CaCl₂) (0.220 g), KCl (0.370 g), KH₂PO₄ (0.03 g), MgCl₂ (0.210 g, MgSO₄ (0.070 g), NaCl (8.000 g), NaHCO₃(0.227 g), Na₂HPO₄ (0.120 g), D-glucose (1.000 g) in 1 L distilled water] for 10 min at RT to lyse any red blood cells. Cell were centrifuged at 350×g for 10 min at 10° C. and the supernatants were discarded and replaced with AIM V (Gibco, Life Technologies) plus 10% Fetal Bovine Serum (FBS; Gibco, Life Technologies). Cells were counted manually using a hemocytometer and Trypan Blue reagent (Gibco, Life Technologies) using standard techniques. Splenocytes were diluted with prewarmed AIM V (Gibco) and 10% FBS (Gibco) to a density of 1.0×10⁶ cells per well (ELISA) into 96 well tissue culture plates (Thermofisher) and placed in a 37° C., 5% CO₂ incubator for an hour to stabilize before stimulation with OVA or media (vaccine trial) or stimulation with adjuvants to measure effect of the immunostimulants on primary splenocytes.

Lymphocyte proliferative response assay: Cells were cultured in 96-well flat-bottom plates (Nalge Nunc International, Naperville, Ill., USA) at 1×10⁵ cells/well and 10% CD14⁺ myeloid cells (as indicated) in a final volume of 200 μl culture medium with triplicate wells. Cells were incubated for 72 h followed by the addition of 0.4 μCi ³H-thymidine (Amersham Pharmacia Biotech/well for another 16 h of culture. Cells were freeze-thawed and harvested onto Unifilter plates (Perking Elmer, Boston, Mass., USA) and incorporation of ³H-thymidine was measured as counts per minute (cpm) using a liquid scintillation counter (Top-Count, Perking Elmer). Each experiment was performed separately with cells isolated from several animals as indicated.

Statistical Analysis: Statistical analyses were carried out using Graph-Pad Prism 6 software (GraphPad Software, San Diego, Calif., USA). Differences in the cytokine production were identified using a non-parametric Kruskal-Wallis ANOVA test where Dunn's multiple comparisons test was used post-hoc to identify statistically significant differences in cytokine production. Differences in the frequency of OVA-stimulated or unstimulated CD4+ T cells were determined using Wilcoxon t tests and differences across treatments were determined using Kruskal-Wallis ANOVA as above. Differences were considered statistically significant at p<0.05 (*), p<0.01 (**), p<0.001 (***) and p<0.0001 (****) as stated in the text.

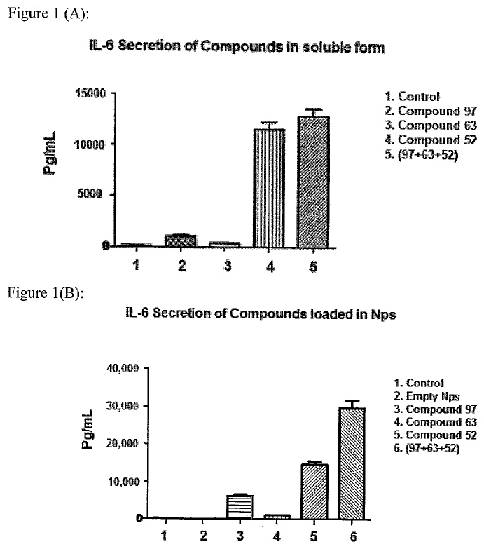

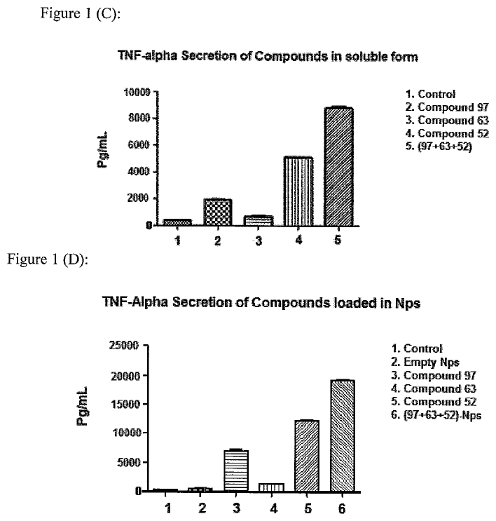

FIGS. 1A-1D shows the results of in vitro assays. Clearly, the immune responses are very strong when the compounds are formulated into PLGA nanoparticles.

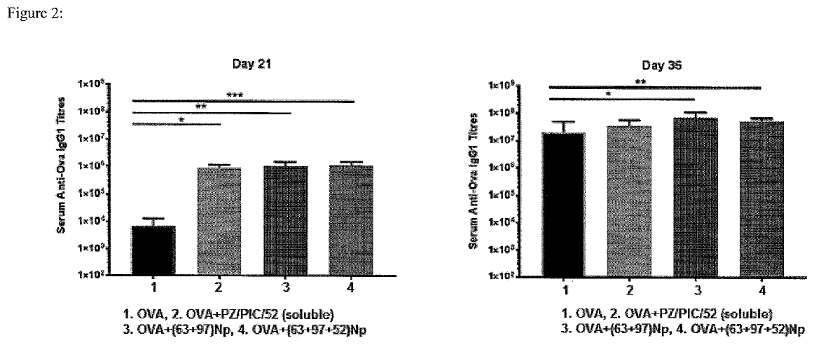

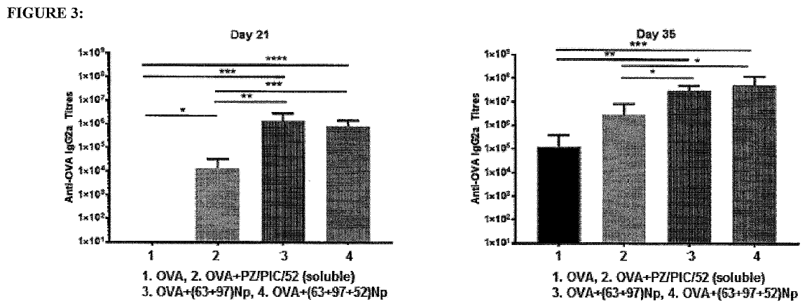

As shown in FIGS. 2 and 3, mice vaccinated with OVA and various combinations of compounds including compounds 52, 63, 97 showed a significant increased production of IgG2a and IgG1.

These results also indicate that these compounds can be used as universal adjuvants for any vaccine that is expected to elicit cellular responses (in therapeutic vaccines) and/or antibody responses (preventive vaccines).

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “between”, “up to”, “at least”, “greater than”, “less than”, “more than”, “or more”, and the like, include the number(s) recited and such terms refer to ranges that can be subsequently broken down into sub-ranges as discussed above.

What is claimed is:

1. An adjuvant comprising a compound of Formula (I): 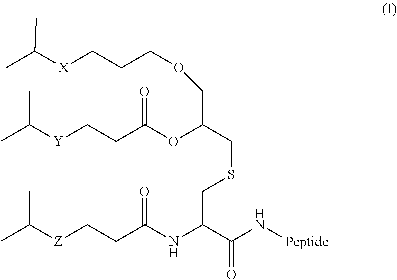
wherein X, Y, and Z are each a spacer, and wherein the peptide comprises Ser, Lys, Phe, Leu, Met, Asp, Gln, and Ala, or any combination thereof, in any sequence including multiple repeats of any single amino acid.

2. (canceled)

3. (canceled)

4. (canceled)

5. (canceled)

6. The adjuvant of claim 1, wherein the peptide comprises one peptide or two peptides separated by a linker.

7. The adjuvant of claim 6, wherein the peptide comprises two peptides separated by a linker comprising an alkylene (CH₂)ₙ, where n is an integer 1≤n≤20.

8. The adjuvant of claim 7, wherein 2≤n≤10.

9. The adjuvant of claim 6, wherein the peptide comprises two peptides separated by a linker and a first peptide comprises 4 to 7 amino acids.

10. The adjuvant of claim 9, where the first peptide comprises Ser, Lys, Asp, Ala, Gln and Lys, or any combination thereof, in any sequence including multiple repeats of any single amino acid.

11. The adjuvant of claim 9, wherein the first amino acid of the first peptide is Ser.

12. The adjuvant of claim 9, wherein the last amino acid of the first peptide is Lys.

13. The adjuvant of claim 6, wherein a second peptide comprises a chemotactic peptide.

14. The adjuvant of claim 13, wherein the chemotactic peptide is a tripeptide comprising Phe, Leu and formyl Met.

15. The adjuvant of claim 1 which is a compound of one of Formula I.1, I.2, I.3 or I.4: 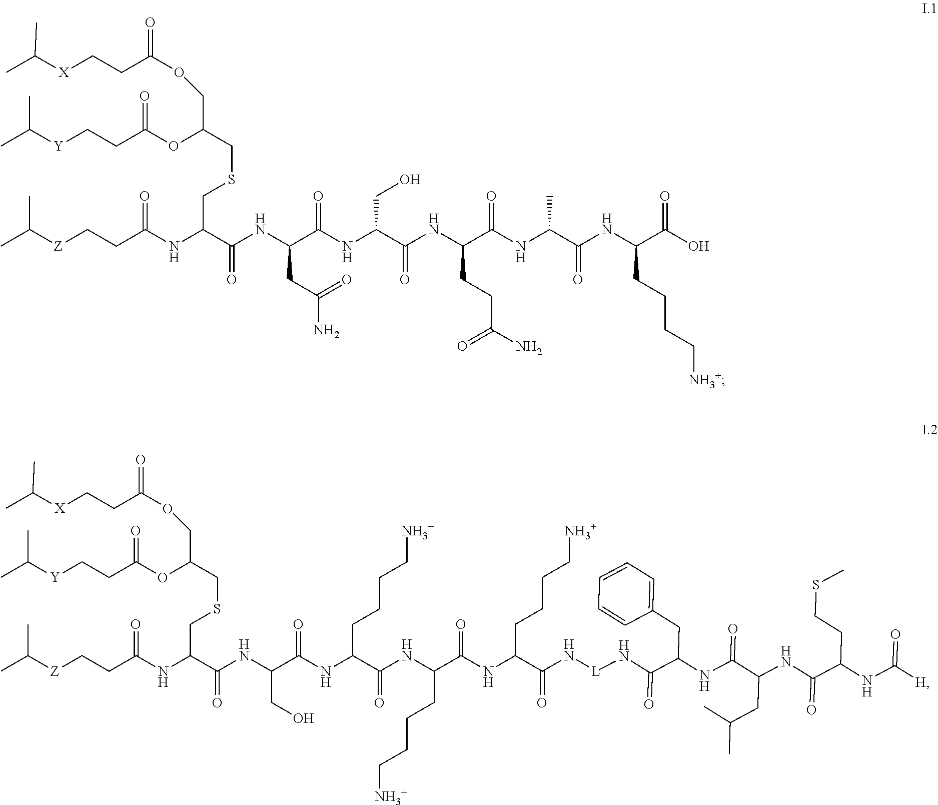
wherein L is a linker;

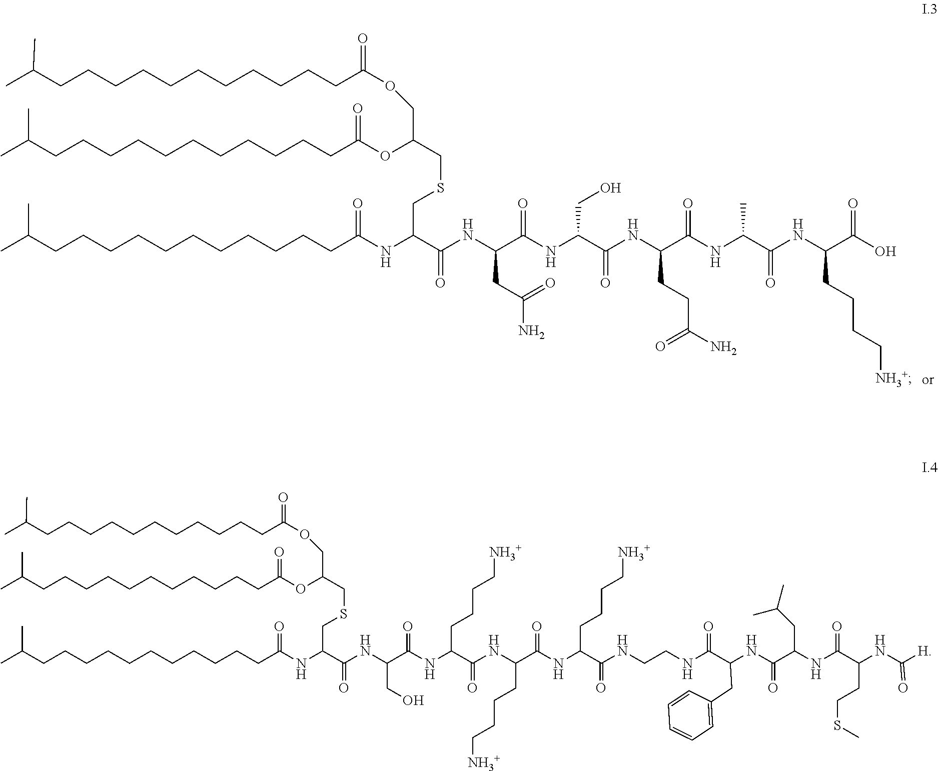

16. An adjuvant comprising a compound of Formula (II): 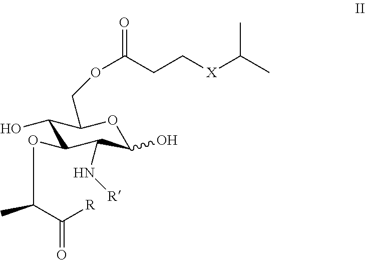
wherein X is a linker, R is a dipeptide or tripeptide and R′ is either an acetyl or a glycolic group.

17. The adjuvant of claim 16, wherein X is substituted or unsubstituted alkylene linker comprising between 6 to 20 carbon atoms.

18. (canceled)

19. (canceled)

20. (canceled)

21. The adjuvant of claim 16 wherein R is a tripeptide comprising Ala, IsoGln and Ser.

22. The adjuvant of claim 16 which is a compound of Formula II.1 or Formula II.2:

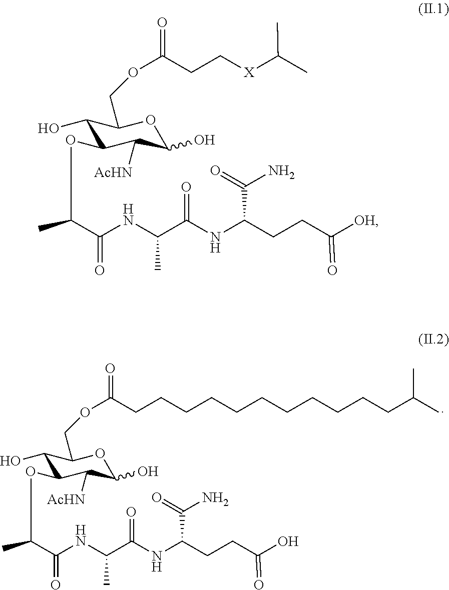

23. (canceled)

24. (canceled)

25. (canceled)

26. (canceled)

27. (canceled)

28. (canceled)

29. An adjuvant formulation comprising a combination of at least two different adjuvants selected from the group consisting of a monophosphoryl Lipid A (MPLA) analogue, a Pam3CSK4 analogue, and a muramyldipeptide (MDP) analogue.

30. The adjuvant formulation of claim 29 wherein the combination of at least two adjuvants is a combination of compounds of Formula I, Formula II or Formula III.

31. The adjuvant formulation of claim 30, comprising a compound a compound of Formula II.2 and a compound of Formula III.7.

32. An adjuvant formulation comprising nanoparticles bearing at least one adjuvant of any one of claim 1, 16 or 29.

33. The adjuvant formulation of claim 32 wherein the nanoparticles comprise polylactic-co-glycolic acid (PLGA) nanoparticles.

34. (canceled)

35. (canceled)

36. A method of vaccinating an animal by administering a vaccine formulation comprising an immunogen and an adjuvant of claim 1 and/or 16, or an adjuvant formulation of claim 29.
